(19) United States
(12) Patent Application Publication
(10) Pub. No.: US 2021/0046014 A1
(43) Pub. Date: Feb. 18, 2021

(54) PHARMACEUTICAL ABUSE DETERRENT COMPOSITION CONSTRUCTED IN MORE THAN ONE STRENGTHS

(71) Applicant: Jayendrakumar Dasharathlal PATEL, Vijapur (IN)

(72) Inventor: Jayendrakumar Dasharathlal PATEL, Vijapur (IN)

(73) Assignee: Jayendrakumar Dasharathlal Patel, Vijapur, OT (IN)

(21) Appl. No.: 16/097,980

(22) PCT Filed: Jun. 16, 2018
(86) PCT No.: PCT/IB2018/054443

§ 371 Date: Oct. 31, 2018

(30) Foreign Application Priority Data
Oct. 1, 2017 (IN) 201727034838

(51) Int. Cl. A61K 9/50; A61K 9/20; A61K 9/28; A61K 9/16

(52) U.S. Cl. CPC A61K 9/501; A61K 9/2013; A61K 9/2031; A61K 9/2866; A61K 9/2853; A61K 9/5026; A61K 9/1641; A61K 9/1652; A61K 9/5047; A61K 9/5015; A61K 9/2009

(57) ABSTRACT

A pharmaceutical abuse deterrent composition and method of making the same is described in the present invention. A pharmaceutical abuse deterrent composition is constructed in one strength or more than one strength, wherein each strength in alone or in combination with another strength has a unique overdose prevention features. A pharmaceutical abuse deterrent composition is constructed in one strength or more than one strength, wherein each strength has resistant to physical tampering and chemical tampering.

### FIELD OF THE INVENTION

The present invention relates to a pharmaceutical abuse deterrent composition and method of making the same.

### BACKGROUND OF THE INVENTION

Abuse of prescription drug is a serious public health problem that affects almost every community and family in some way. Each year drug abuse causes millions of serious illnesses or injuries among the peoples. Drug abuse also plays a role in many major social problems, such as drugged driving, violence, stress, and child abuse. Drug abuse can lead to homelessness, crime, and missed work or problems with keeping a job. It also harms unborn babies and destroys families. There are different types of treatment for drug abuse. But the best is to reduce the drug abuse in the first place by making abuse deterrent pharmaceutical composition.

Some common techniques for intentionally abusing a drug begin with an abuser is obtaining a solid dosage form such as an orally administered tablet or capsule, and crushing the solid dosage form into a powder. The powder may be administered by an abuser by nasal insufflation (i.e., “snorting”) to introduce the drug to the abuser's bloodstream intranasally. Alternately, the crushed dosage form may be combined with a solvent that is capable of dissolving the drug substance, and the solvent with the dissolved drug substance may be injected directly into an abuser's bloodstream. This type of administration results in an even faster diffusion of the drug substance compared to the oral abuse, with the result desired by the abuser, namely the kick.

Alternatively, with immediate release or modified release oral dosage forms, an abuser might simply ingest multitude of the dosage form (e.g., tablets or capsule) together, e.g., simultaneously. Each one of the multiple dosage form units produce a short-term or a long-term concentration spike of the drug in the user's bloodstream.

Various concepts for the avoidance of drug abuse have been developed by making abuse deterrent dosage form. The requirements for abuse deterrent dosage forms that nowadays need to be satisfied are complex and sometimes are difficult to be combined and arranged with one another. While a certain measure may improve abuse deterrent in a certain aspect, the same measure may deteriorate abuse deterrent in another aspect or otherwise may have a detrimental effect on the properties of the dosage forms.

Although the pharmaceutical industry has identified a variety of abuse deterrent features useful with oral dosage forms, still there is continuing need to improve and identify new abuse deterrent features to inhibit or prevent abuse or overdosing of active ingredients.

It is therefore an object of the present invention is to construct a pharmaceutical abuse deterrent composition that has a unique overdose prevention feature and at least resistant to physical tampering and chemical tampering.

### SUMMARY OF THE INVENTION

An object of the present invention is to construct a pharmaceutical abuse deterrent composition in one strength or more than one strength, wherein each strength in alone or in combination with another strength has a unique overdose prevention features.

In preferred embodiment, a pharmaceutical abuse deterrent composition containing particulates in which a release of a drug substance is controlled,
wherein the composition is constructed in more than one strength which comprises a first strength having a first acid neutralisation capacity and a second strength having a second acid neutralisation capacity, each of the first strength and the second strength individually contain an acid neutralising ingredient and the particulates of the drug substance, wherein
   a. an amount of the particulates of the drug substance in the first strength is at least about 10% higher relative to the second strength and
   b. the second acid neutralisation capacity of the second strength is lower than the first acid neutralisation capacity of the first strength.

In preferred embodiment, each of the first strength and the second strength is administered individually to treat a disease condition of a subject in need thereof

Another object of the present invention is to construct a pharmaceutical abuse deterrent composition which is at least resistant to physical or chemical tampering.

In preferred embodiment, a pharmaceutical abuse deterrent composition comprising coated particulates, wherein each coated particulate comprising
a. a particulate of anti-crushing ingredient having particle size in the range of 50 micron to 1500 micron, wherein the anti-crushing ingredient is gelling agent and present in amount of not less than about 10% w/w of the composition,
b. a first coat surrounding the particulate and comprising at least one of a water insoluble ingredient, pH independent ingredient and pH dependent ingredient, and
c. an outer coat surrounding the first coat and comprising an active ingredient;
wherein less than about 10% the said coated particulates pass through #200 mesh sieve; and
wherein the coated particulates exhibit breaking strength at least sufficient to resist pulverisation, thereby less than about 40% pulverized particles of the coated particulates pass through #400 mesh sieve when subjected to the coffee grinder for about 1 minute.

### DETAILED DESCRIPTION OF THE INVENTION

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing”, “comprising” and “comprises” are interchangeable, and one of skill in the art is cognizant that these terms are open-ended terms.

The term “pharmaceutical abuse deterrent composition” or “abuse deterrent pharmaceutical composition” is used to refer to a composition that deter the potential for abuse but delivers a therapeutically effective amount of a drug substance when administered as directed. For example, these terms refer to a composition that can be at least resistant to physical or chemical tampering. The term physical tampering includes, with or without heat treatment or freezing, at least one of crushing, grinding, melting, cutting, and like. The term chemical tampering includes, extracting the drug substance from the composition, dose dumping (e.g., alcohol dose dumping), and solubilizing the composition for injection purposes. The abuse deterrent composition deters an improper administration of the composition or drug substance contained in the composition, wherein improper administration includes, without limitation, administering the drug substance or the composition with 40% w/w alcohol, administering the drug substance or the composition by any route other than that instructed after physical or chemical tampering with the composition. For example, without limitation, improper administration includes snorting after grinding, administration after heat treatment, oral administration after crushing, or parenteral administration after extraction of the drug substance from the composition with a solvent, and the like, and combinations thereof. The abuse deterrent composition diminishes the drug substance release after accidental or intentional simultaneous administration of a plurality of dosage units of the composition containing an overall supratherapeutic dose of the drug substance. The term “supratherapeutic dose” refer to the overall content of the drug substance originally contained in the plurality of dosage units of the composition is greater than two or more time of the highest strength or greater than maximum daily recommended dose of the drug substance. In other word, a pharmaceutical abuse deterrent composition has overdose protection feature. The term “overdose protection” refers to a feature of dosage form that reduces the potential for the detrimental consequences of overdose but delivers a therapeutically effective dose when administered as directed or prescribed by a physician.

The term “dosage form” or “dosage unit” or “strength” are used interchangeably to refer to oral drug delivery system of the pharmaceutical abuse deterrent composition comprising a predetermined quantity (therapeutically effective dose) of one or more active ingredient(s) and suitable as unitary dosages for administration to human patients to attain the desired therapeutic effect.

In preferred embodiment, a pharmaceutical abuse deterrent composition deliver a drug substance in at least one of immediate release form and modified release form.

The term “immediate release” refers to dosage form that are formulated to allow the drug to dissolve in the gastrointestinal contents or in dissolution media with no intention of delaying or prolonging the absorption when taken as prescribed or the dissolution of the drug substance when subjected to in vitro dissolution testing.

The term “modified release” refers to dosage forms that are formulated to allow the drug to dissolve at slower rate in the gastrointestinal contents or in dissolution media over a greater period of time, e.g. for period of 4 hours, 8 hours, 12 hours, 16 hours, 20 hours and like, as compared to a drug presented as a conventional dosage form (e.g., immediate release). The term “modified release” also include release of the drug substance at any rate after the predetermined lag time or in the predetermined location of gastrointestinal tract after administration.

As used herein, use of phrases such as “diminish” or “lower” is meant to include at least a 10% change in, e.g., the release rate of an active ingredient or amount of the drug substance. With respect to “the release rate of an active ingredient”, greater percentage changes being preferred for reduction in overdose potential. For example, but without limitation, the change may be greater than 25%, 35%, 45%, 55%, 65%, 75%, 85%, 95%, 96%, 97%, 98%, 99%, or increments therein.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, preferably within five-fold, and more preferably within two-fold, of a value.

The term “in-vitro dissolution testing” refers to in-vitro test that measure the rate and extent of release of the drug substance from dosage unit(s) of the composition at 37°±1° C. in 900 ml 0.01N HCl in a USP Apparatus 2 (paddle) at 50 rpm and optionally with sinker.

The “wt. %” or “% wt.” refers to percentage by weight.

In preferred embodiment, a pharmaceutical abuse deterrent composition containing particulates in which a release of a drug substance is controlled,
wherein the composition is constructed in more than one strength which comprises a first strength having a first acid neutralisation capacity and a second strength having a second acid neutralisation capacity, each of the first strength and the second strength individually contain an acid neutralising ingredient and the particulates of the drug substance, wherein
   a. an amount of the particulates of the drug substance in the first strength is at least about 10% higher relative to the second strength, and
   b. the second acid neutralisation capacity of the second strength is lower than the first acid neutralisation capacity of the first strength,
wherein the release of the drug substance in each particulate is controlled by a pH dependent ingredient depending on pH of surrounding environment which is raise or maintained by the acid neutralising ingredient, wherein
   a. the pH dependent ingredient is present in the each particulate of drug substance as
      1) matrixing agent in which the drug substance is embedded, or
      2) an coating layer surrounding the particulate containing the drug substance, or
      3) a combination of the step 1) and the step 2), and
   b. the acid neutralising ingredient is present as separate particulates or coated on the particulates of drug substance,
wherein the first strength and the second strength being adapted to prevent an overdose of the drug substance after accidental or intentional simultaneous administration of a multitude of the first strengths or the second strengths or combination thereof containing an overall supratherapeutic dose of the drug sub stance.

In certain embodiment, the particulate of the drug substance further comprising a functional ingredient to attain modified release profile of the drug substance, wherein the functional ingredient is at least one of pH independent ingredient and water insoluble ingredient. In preferred embodiment, the functional ingredient is present in the each particulate of drug substance as
1) matrixing agent in which the drug substance is embedded, or
2) an coating layer surrounding the particulate containing the drug substance, or
3) a combination of the step 1) and the step 2)

In preferred embodiment, a pharmaceutical abuse deterrent composition containing particulates in which a release of a drug substance is controlled,
wherein the composition is constructed in more than one strength which comprises a first strength and a second strength, each of the first strength and the second strength individually contain an acid neutralising ingredient and the particulates of the drug substance, wherein
   a. an amount of the particulates of the drug substance in the first strength is at least about 10% higher relative to the second strength, and
   b. an amount of the acid neutralising ingredient in the second strength is at least about 5% lower relative to the first strength,
wherein the release of the drug substance in each particulate of each strength is controlled by a pH dependent ingredient depending on pH of surrounding environment which is raise or maintained by the acid neutralising ingredient, wherein
   a. the pH dependent ingredient is present in the each particulate of drug substance as
      1) matrixing agent in which the drug substance is embedded, or
      2) an outer coating layer surrounding the particulate containing the drug substance, or
      3) a combination of the step 1) and the step 2), and
   b. the acid neutralising ingredient is present as separate particulates or coated on the particulates of drug substance,
wherein the particulates in a single dosage unit of each strength has similar release profile of the drug substance relative to the other strength but the particulates in a multitude of 3 or more dosage units of each strength has different release profile of the drug substance relative to the other strength when subjected to in-vitro dissolution testing.

A similarity factor F2 value can be used as indicative for evaluating a similarity of release profile of the drug substance from two different strengths, i.e. the first strength and the second strength. A f2 value of 50 or greater (50 to 100) ensures sameness or similarity of the two different release profile. The f2 value is calculated by the following equation:

f2=50×log{[1+(1/n)Σₜ₌₁ⁿ(F_{T}+S_{T})²]^{-0.5}×100}
where Ft and St are the cumulative percentage of the drug substance released at each of the selected n time points of the first strength and the second strength, respectively.

For calculating f2 value,
Mean dissolution values can be used to estimate the similarity factor, f2. To use mean data, the % coefficient of variation at the earlier point should not be more than 20% and at other time points should not be more than 10%.
For circumstances where wide variability is observed, or a statistical evaluation of f2 metric is desired, a bootstrap approach to calculate a confidence interval can be performed.
In vitro dissolution tests are recommended to be performed under the same test conditions.
The dissolution time points for the first strength and the second strength should be the same, for example, for immediate release products e.g. 10, 15, 30, 45, 60, 90 minutes and for extended release products, e.g., 1, 2, 3, 4, 6, 8, 10, 12, 16, 20, and 24 hours.
For release profile comparison, at least three dissolution time points are required.
Because f2 values are sensitive to the number of dissolution time points, only one time point should be considered after 85% release of the drug substance from at least one release profile.
For products which are rapidly dissolving, i.e., more than 85% drug release from dosage unit(s) in 15 minutes or less, a profile comparison is not necessary.

In one embodiment, the first strength is highest strength or lower strength than the highest strength but higher strength than the lowest strength. In another embodiment, the second strength is lowest strength or higher strength than the lowest strength but lower strength than the highest strength.

The term “highest strength” refers to a dosage unit comprising highest amount of active ingredient compared to all other strengths comprising the same active ingredient. The term “lowest strength” refers to a dosage unit comprising lowest amount of active ingredient compared to all other strengths comprising the same active ingredient. For example, unit dosage form of different strength such as 10 mg, 15 mg, 20 mg, 30 mg, wherein 30 mg is highest strength (first strength), 10 mg is lowest strength (second strength), 15 mg and 20 mg can be considered either as a first strength with respect to the lowest strength (10 mg) or as a second strength with respect to the highest strength (30 mg). Furthermore, 15 mg can be considered either as a second strength with respect to 20 mg strength and 30 mg strength or as a first strength with respect to 10 mg strength. Similarly, 20 mg can be considered either as a second strength with respect to 30 mg strength or as a first strength with respect to 10 mg strength and 15 mg strength. In other word, 30 mg is first strength, 10 mg is lowest strength, 15 mg or 20 mg can be considered either as first strength or as second strength.

In preferred embodiment, a pharmaceutical abuse deterrent composition containing particulates in which a release of a drug substance is controlled,
wherein the composition is constructed in more than one strength which comprises a first strength having a first acid neutralisation capacity and a second strength having a second acid neutralisation capacity, each of the first strength and the second strength individually contain an acid neutralising ingredient and the particulates of the drug substance, wherein
   a. an amount of the particulates of the drug substance in the first strength is at least about 10% higher relative to the second strength and
   b. the second acid neutralisation capacity of the second strength is lower than the first acid neutralisation capacity of the first strength,
wherein the first strength, the second strength and the multitude of the second strength when subjected to in-vitro dissolution testing for at least one corresponding time point:
a) the second strength has released more than about 70% wt. of the drug substance content originally contained in the second strength,
b) a multitude of the second strength has released more than about 70% wt. of the overall content of the drug substance originally contained in the multitude of the second strength, wherein the overall content of the drug substance originally contained in the multitude of the second strength is greater than the content of the drug substance originally contained in the first strength but lower than the supratherapeutic dose of the drug substance, and
c) a multitude of the second strength has released less than about 70% wt. of the overall content of the drug substance originally contained in the multitude of the second strength, wherein the overall content of the drug substance originally contained in the multitude of the second strength is greater than the supratherapeutic dose of the drug substance.

In preferred embodiment, pharmaceutical abuse deterrent composition containing particulates in which a release of a drug substance is controlled,
wherein the composition is constructed in more than one strength which comprises a first strength having a first acid neutralisation capacity and a second strength having a second acid neutralisation capacity, each of the first strength and the second strength individually contain an acid neutralising ingredient and the particulates of the drug substance, wherein
   a. the second acid neutralisation capacity of the second strength is lower than the first acid neutralisation capacity of the first strength and
   b. an amount of the particulates of the drug substance in the first strength is at least about 10% higher relative to the second strength,
wherein the first strength, the second strength and the multitude of the second strength when subjected to in-vitro dissolution testing for at least one corresponding time point:
   a) difference between the drug substance release from the first strength and the drug substance release from the second strength is less than about 25,
   b) difference between the drug substance release from the first strength and the drug substance release from a multitude of the second strength is less than about 25, wherein the overall content of the drug substance originally contained in the multitude of the second strength is greater than the content of the drug substance originally contained in the first strength but lower than the supratherapeutic dose of the drug substance, and
   c) difference between the drug substance release from the first strength and the drug substance release from a multitude of the second strength is more than about 25, wherein the overall content of the drug substance originally contained in the multitude of the second strength is equal to or greater than the supratherapeutic dose of the drug substance,
wherein the drug substance released from the first strength, the second strength and the multitude of the second strength is measured in % wt. of the overall content of the drug substance originally contained in the first strength, the second strength and the multitude of the second strength, respectively.

In preferred embodiment, a pharmaceutical abuse deterrent composition containing particulates in which a release of a drug substance is controlled,
wherein the composition is constructed in more than one strength which comprises a first strength having a first acid neutralisation capacity and a second strength having a second acid neutralisation capacity, each of the first strength and the second strength individually contain an acid neutralising ingredient and the particulates of the drug substance, wherein an amount of the particulates of the drug substance in the first strength is at least about 10% higher relative to the second strength,
wherein each of the first strength and the second strength individually contain a therapeutic effective dose of the drug substance to raise a plasma level of the drug substance in a mammal to whom it is administered such that
   1. a single dose Cmax of the second strength after administration is dose proportional with respect to the corresponding single dose Cmax of the first strength after administration, and
   2. a single dose Cmax of a multitude of the second strength after administration is less than the minimum level for dose proportionality with respect to the corresponding single dose Cmax of the first strength after administration, wherein the overall content of the drug substance originally contained in the multitude of the second strength is higher than the supratherapeutic dose of the drug substance.

In preferred embodiment, a pharmaceutical abuse deterrent composition containing particulates in which a release of a drug substance is controlled,
wherein the composition is constructed in more than one strength which comprises a first strength having a first acid neutralisation capacity and a second strength having a second acid neutralisation capacity, each of the first strength and the second strength individually contain an acid neutralising ingredient and the particulates of the drug substance, wherein
   a. an amount of the particulates of the drug substance in the first strength is at least about 10% higher relative to the second strength and
   b. the second acid neutralisation capacity of the second strength is lower than the first acid neutralisation capacity of the first strength, and
wherein each of the first strength and the second strength individually contain a therapeutic effective dose of the drug substance to raise a plasma level of the drug substance in a mammal to whom it is administered such that
   1. a single dose Cmax of the second strength after administration is dose proportional with respect to the corresponding single dose Cmax of the first strength after administration, and
   2. a single dose Cmax of a multitude of the second strength after administration is less than the minimum level for dose proportionality with respect to the corresponding single dose Cmax of the first strength after administration, wherein the overall content of the drug substance originally contained in the multitude of the second strength is higher than the supratherapeutic dose of the drug substance.

The pharmacokinetic parameter, namely Cmax, considered dose proportional if increasing approximately proportionally with respect to increasing the amount of the drug substance. The term “approximately proportionally” means±20% of the reference value. The “less than the minimum level for dose proportionality” means less than 80% of reference value. In the present invention, value of single dose Cmax of the first strength is the reference value.

According to invention, the acid neutralisation capacity of the first strength and the second strength can be measure by following method (perform the following procedure at 37°±1° C. in USP apparatus II (Paddle) at 250 RPM and optionally with sinker):
1. Take 900 ml aqueous media containing 0.01N HCl (HCl diluted with purified water) in two separate dissolution vessels and denote the vessel with “V1” for first vessel and “V2” for second vessel.
2. Measure initial pH of the aqueous media of both vessels. Difference of the measured pH of the aqueous media of the vessel “V1” and the vessel “V2” should not be more than about 0.15.
3. Add one by one a dosage unit of the first strength at interval of 15 minutes into the vessel “V1” and measured the pH of the aqueous media after 15 minutes of addition of each dosage unit. Continuously add a dosage unit of the first strength one by one till pH of the aqueous media elevate at least about 5.5. Measure the final pH (A) of the aqueous media after 30 minutes of addition of last dosage unit of the first strength.
4. Add one by one a dosage unit of the second strength at interval of 15 minutes into the vessel “V2”, wherein total number of dosage units of the second strength to be added in the vessel “V2” is equal to the number of the dosage units of the first strength added in the vessel “V1”. Measure the final pH (B) of the aqueous media after 30 minutes of addition of last dosage unit of the second strength.
5. Calculate the difference of the final pH (X) as follow:

Difference of the final pH(X)=the final pH(A)−the final pH(B)

Acid neutralisation capacity of the first strength is considered higher than acid neutralisation capacity of the second strength when the value of the final pH (X) is positive value (+).

In preferred embodiment, the particulates of the drug substance in each of the first strength and the second strength comprising more than one matrix particulates, wherein the each matrix particulate comprising
1) a particulate comprising the drug substance and a pH dependent ingredient, wherein the drug substance is dispersed within matrix of the pH dependent ingredient, wherein the pH dependent ingredient is present in amount of not less than about 50% w/w of an amount of the drug substance and is at least one of
   a) polyvinylacetal diethylamino acetate,
   b) copolymer of dimethylaminoethyl methacrylate, butyl methacrylate, methyl methacrylate, and
   c) copolymer of diethylaminoethyl methacrylate and methyl methacrylate, and
2) optionally an outer coating layer surrounding the particulate, wherein the outer coating layer comprising at least one of pH independent ingredient, water insoluble ingredient, surfactant, stabilizer, plasticizer and anti-tacking ingredient.

In preferred embodiment, the particulates of the drug substance in each of the first strength and the second strength comprising a more than one coated particulates, wherein the each coated particulate comprising
1) a particulate comprising coat of the drug substance surrounding at least one of gelling ingredient, anti-crushing ingredient, pH independent ingredient, water insoluble ingredient and the particulate of aversion agent, and
2) an outer coating layer of the pH dependent ingredient surrounding the particulate, wherein the outer coating layer optionally further comprising at least one of pH independent ingredient, water insoluble ingredient, surfactant, stabilizer, plasticizer and anti-tacking ingredient, wherein the pH dependent ingredient is present in amount of not less than about 10% w/w of an amount of the drug substance and is at least one of
   a) polyvinylacetal diethylamino acetate,
   b) copolymer of dimethylaminoethyl methacrylate, butyl methacrylate, methyl methacrylate, and
   c) copolymer of diethylaminoethyl methacrylate and methyl methacrylate.

In preferred embodiment, the particulates of the drug substance in each of the first strength and the second strength comprising more than one coated particulates, wherein the each coated particulate comprising
1) a particulate comprising coat of the drug substance surrounding at least one of gelling ingredient, anti-crushing ingredient, pH independent ingredient, water insoluble ingredient and the particulate of aversion agent,
2) a functional coat surrounding the particulate and comprising at least one of pH independent ingredient, water insoluble ingredient, surfactant, stabilizer, plasticizer and anti-tacking ingredient, and
3) an outer coating layer of the pH dependent ingredient surrounding the functional coat, wherein the outer coating layer optionally further comprising at least one of pH independent ingredient, water insoluble ingredient, surfactant, stabilizer, plasticizer and anti-tacking ingredient, wherein the pH dependent ingredient is present in amount of not less than about 10% w/w of an amount of the drug substance and is at least one of
   a) polyvinylacetal diethylamino acetate,
   b) copolymer of dimethylaminoethyl methacrylate, butyl methacrylate, methyl methacrylate, and
   c) copolymer of diethylaminoethyl methacrylate and methyl methacrylate.

In preferred embodiment, the particulates of the drug substance in each of the first strength and the second strength comprising more than one coated particulates, wherein the each coated particulate comprising
1) a particulate comprising the drug substance and at least one of pH dependent ingredient, pH independent ingredient, water insoluble ingredient, anti-crushing ingredient, and gelling ingredient, wherein the drug substance is dispersed within matrix of at least one of pH dependent ingredient, pH independent ingredient, water insoluble ingredient, anti-crushing ingredient, and gelling ingredient, and
2) the outer coating layer of the pH dependent ingredient surrounding the particulate, wherein the outer coating layer optionally further comprising at least one of pH independent ingredient, water insoluble ingredient, surfactant, stabilizer, plasticizer and anti-tacking ingredient, wherein the pH dependent ingredient is present in amount of not less than about 10% w/w of an amount of the drug substance and is at least one of
   a) polyvinylacetal diethylamino acetate,
   b) copolymer of dimethylaminoethyl methacrylate, butyl methacrylate, methyl methacrylate, and
   c) copolymer of diethylaminoethyl methacrylate and methyl methacrylate.

In preferred embodiment, the particulates of the drug substance in each of the first strength and the second strength comprising more than one coated particulates, wherein the each coated particulate comprising
1) a particulate comprising the drug substance and at least one of pH dependent ingredient, pH independent ingredient, water insoluble ingredient, anti-crushing ingredient, and gelling ingredient, wherein the drug substance is dispersed within matrix of at least one of pH dependent ingredient, pH independent ingredient, water insoluble ingredient, anti-crushing ingredient, and gelling ingredient,
2) a functional coat surrounding the particulate and comprising at least one of pH independent ingredient, water insoluble ingredient, surfactant, stabilizer, plasticizer and anti-tacking ingredient, and
3) the outer coating layer of the pH dependent ingredient surrounding the functional coat, wherein the outer coating layer optionally further comprising at least one of pH independent ingredient, water insoluble ingredient, surfactant, stabilizer, plasticizer and anti-tacking ingredient, wherein the pH dependent ingredient is present in amount of not less than about 10% w/w of an amount of the drug substance and is at least one of
   a) polyvinylacetal diethylamino acetate,
   b) copolymer of dimethylaminoethyl methacrylate, butyl methacrylate, methyl methacrylate, and
   c) copolymer of diethylaminoethyl methacrylate and methyl methacrylate.

In preferred embodiment, the particulates of the drug substance in each of the first strength and the second strength comprising more than one coated particulates, wherein the each coated particulate comprising
a. a particulate comprising a pharmaceutically acceptable derivative of the drug substance; and
b. the outer coating layer of the pH dependent ingredient surrounding the derivatives of drug substance, wherein the outer coating layer optionally further comprising at least one of pH independent ingredient, water insoluble ingredient, surfactant, stabilizer, plasticizer and anti-tacking ingredient.

In preferred embodiment, the particulates of the drug substance in each of the first strength and the second strength comprising more than one matrix particulates, wherein the each matrix particulate comprising a pharmaceutically acceptable derivative of the drug substance and a pH dependent ingredient, wherein the derivatives of drug substance is dispersed within matrix of the pH dependent ingredient, wherein the pH dependent ingredient is present in amount of not less than about 50% w/w of an amount of the drug substance and is at least one of
a) polyvinylacetal diethylamino acetate,
b) copolymer of dimethylaminoethyl methacrylate, butyl methacrylate, methyl methacrylate, and
c) copolymer of diethylaminoethyl methacrylate and methyl methacrylate.

In preferred embodiment, the particulates of the drug substance in each of the first strength and the second strength further comprising at least one ingredient of pH independent ingredient, ion-exchange resin, water insoluble ingredient, gelling ingredient, anti-crushing ingredient, diluent, glidant, surfactant, stabilizer, binder, lubricant, disintegrant, plasticizer, anti-tacking agent, surfactant, and stabilizer.

In preferred embodiment, each of the first strength and the second strength individually further containing particulates of an aversion agent, wherein the aversion agent is not released in effective amount to produce its intended aversive effect unless the composition is administered after physical alteration but is released in effective amount to produce its intended aversive effect when the composition is administered after the physical alteration. Physical alteration includes crushing, grinding, cutting and like. Thus, the particulates of an aversion agent deter an abuse from physical tampering and chemical tampering. The aversion agent is present as coated particulates in each of the first strength and the second strength, wherein each coated particulate comprising
1) a particulate comprising aversion agent, wherein the aversion agent is present
   a) as a particle or
   b) within matrix of at least one ingredient of pH dependent ingredient, pH independent ingredient and water insoluble ingredient or
   c) as a coat surrounding at least one of pH dependent ingredient, pH independent ingredient and water insoluble ingredient, and
2) water insoluble coat surrounding the particulate.

In certain embodiment, the particulates of the drug substance in each of the first strength and the second strength further comprising barrier coat before and/or after the outer coat of the pH dependent ingredient, wherein the barrier coat comprising at least one of pH independent ingredient, water insoluble ingredient, surfactant, stabilizer, plasticizer and anti-tacking ingredient.

In certain embodiment, the particulates of the first strength and/or the second strength further comprising functional coat before and/or after the outer coat of the pH dependent ingredient to attain modified release profile, wherein the functional coat comprising at least one of pH independent ingredient, water insoluble ingredient, surfactant, stabilizer, plasticizer and anti-tacking ingredient.

In preferred embodiment, acid neutralisation capacity of the first strength and/or the second strength is attained by the acid neutralising ingredient.

In preferred embodiment, the acid neutralising ingredient can be present as separate particulates in the dosage unit or coated on the particulates of the drug substance. In one embodiment, the acid neutralising ingredient is present as the separate particulates in the dosage unit, wherein each particulate comprising the acid neutralising ingredient, and optionally, at least one of diluent, glidant, binder, lubricant and disintegrant.

In one embodiment, the pH dependent ingredient has the pH dependent solubility, wherein it is soluble in stomach fluid or aqueous fluid at pH below about 5 but substantially insoluble in intestinal fluid or in stomach fluid or in aqueous fluid at pH above about 5.5. In another embodiment, the acid neutralisation capacity of the first strength and/or the second strength retard the solubilisation of the pH dependent ingredient in stomach fluid or in aqueous fluid by elevating the pH of stomach fluid or the aqueous fluid above about 5.5 as a function of the amount of the pharmaceutical composition ingested: when the first strength and/or the second strength is ingested in an appropriate amount, the acid neutralisation capacity of the first strength and/or the second strength not sufficiently raise the pH of stomach fluid or aqueous fluid above about pH 5.5, and the pH dependent ingredient dissolves and releases the drug substance; when the first strength and/or the second strength is ingested in an excess amount, the acid neutralisation capacity of the first strength and/or the second strength elevate the gastrointestinal pH above about 5.5, thereby preventing the pH dependent ingredient from dissolving and releasing the drug substance.

When the second strength or a multitude of the second strength subjected to the in-vitro dissolution testing, the content of the acid neutralising ingredient originally contained in the second strength or the overall content of the acid neutralising ingredient originally contained in the multitude of the second strength not elevate the pH of 0.01N HCl above about 5.5, wherein the content of the drug substance originally contained in the second strength or the overall content of the drug substance originally contained in the multitude of the second strength is lower than the supratherapeutic dose of the drug substance, respectively.

When a multitude of the second strength subjected to the in-vitro dissolution testing, the overall content of the acid neutralising ingredient originally contained in the multitude of the second strength elevate the pH of 0.01N HCl above about 5.5, wherein the overall content of the drug substance originally contained in the multitude of the second strength is equal or greater than the supratherapeutic dose of the drug substance.

When the combination dosage units of the second strength(s) and the first strength(s) subjected to the in-vitro dissolution testing, the overall content of the acid neutralising ingredient originally contained in the combination dosage units not elevate the pH of 0.01N HCl above about 5.5, wherein the overall content of the drug substance originally contained in the combination dosage units is lower than the supratherapeutic dose of the drug substance.

When the combination dosage units of the second strength(s) and the first strength(s) subjected to the in-vitro dissolution testing, the overall content of the acid neutralising ingredient originally contained in the combination dosage units elevate the pH of 0.01N HCl above about 5.5, wherein the overall content of the drug substance originally contained in the combination dosage units is equal or greater than the supratherapeutic dose of the drug sub stance.

In preferred embodiment, a pharmaceutical abuse deterrent composition manufactured by non-thermal process comprising coated particulates, wherein each coated particulate comprising
a. a particulate of anti-crushing ingredient having particle size in the range of 50 micron to 1500 micron, wherein the anti-crushing ingredient is gelling agent and present in amount of not less than about 10% w/w of the composition, and
b. an outer coat surrounding the particulate and comprising an active ingredient,
wherein less than about 10% the said coated particulates pass through #200 mesh sieve, and
wherein the coated particulates exhibit breaking strength at least sufficient to resist pulverisation, thereby less than about 40% pulverized particles of the coated particulates pass through #400 mesh sieve when subjected to the coffee grinder for about 1 minute.

In preferred embodiment, a pharmaceutical abuse deterrent composition manufactured by non-thermal process comprising coated particulates, wherein each coated particulate comprising
a. a particulate of anti-crushing ingredient having particle size in the range of 50 micron to 1500 micron, wherein the anti-crushing ingredient is gelling agent and present in amount of not less than about 10% w/w of the composition,
b. a first coat surrounding the particulate and comprising at least one of a water insoluble ingredient, pH independent ingredient and pH dependent ingredient, and
c. an outer coat surrounding the first coat and comprising an active ingredient, wherein less than about 10% the said coated particulates pass through #200 mesh sieve, and
wherein the coated particulates exhibit breaking strength at least sufficient to resist pulverisation, thereby less than about 40% pulverized particles of the coated particulates pass through #400 mesh sieve when subjected to the coffee grinder for about 1 minute.

In preferred embodiment, a pharmaceutical abuse deterrent composition manufactured by non-thermal process comprising coated particulates, wherein each coated particulate comprising
a. a particulate of anti-crushing ingredient having particle size in the range of 50 micron to 1500 micron, wherein the anti-crushing ingredient is gelling agent and present in amount of not less than about 10% w/w of the composition,
b. a first coat surrounding the particulate and comprising an active ingredient, and
c. an outer coat surrounding the first coat, wherein the outer coat comprising at least one of a water insoluble ingredient, pH independent ingredient and pH dependent ingredient,
wherein less than about 10% the said coated particulates pass through #200 mesh sieve, and
wherein the coated particulates exhibit breaking strength at least sufficient to resist pulverisation, thereby less than about 40% pulverized particles of the coated particulates pass through #400 mesh sieve when subjected to the coffee grinder for about 1 minute.

In preferred embodiment, a pharmaceutical abuse deterrent composition manufactured by non-thermal process comprising coated particulates, wherein each coated particulate comprising
a. a particulate of anti-crushing ingredient having particle size in the range of 50 micron to 1500 micron, wherein the anti-crushing ingredient is gelling agent and present in amount of not less than about 10% w/w of the composition,
b. a first coat surrounding the particulate and comprising at least one of a water insoluble ingredient, pH independent ingredient and pH dependent ingredient,
c. a second coat surrounding the first coat and comprising an active ingredient, and
d. an outer coat surrounding the second coat, wherein the outer coat comprising at least one of a water insoluble ingredient, pH independent ingredient and pH dependent ingredient,
wherein less than about 10% the said coated particulates pass through #200 mesh sieve, and
wherein the coated particulates exhibit breaking strength at least sufficient to resist pulverisation, thereby less than about 40% pulverized particles of the coated particulates pass through #400 mesh sieve when subjected to the coffee grinder for about 1 minute.

In the present invention, the term “particulates” refers to a discrete, small, repetitive unit of particle, granule, pellet, sphere, bead, mini-tablet, or micro-tablet, that include at least one excipient and, optionally, an active ingredient.

In the present invention, the term “outer coat” refers to a coating, layer, membrane, film, etc. applied to a surface, and, in certain embodiments, can partially, substantially, or completely surround, envelop, cover, enclose, or encase the surface of a particulate to which it is applied. For example, a coat may cover portions of the surface to which it is applied, e.g., as a partial layer, partial coating, partial membrane, or partial film, or the coat may completely cover the surface to which it is applied.

In preferred embodiment, a pharmaceutical abuse deterrent composition is manufacture either by non-thermal process or thermal process. In thermal manufacturing process, heat is employed in at least one stage of manufacturing process of the pharmaceutical composition and temperature of the heat is at least about melting or softening temperature of the anti-crushing ingredient or the pH dependent ingredient used in the pharmaceutical composition. In non-thermal manufacturing process, heat is employed in at least one stage of manufacturing process of the pharmaceutical composition and temperature of the heat is at least about 10% lower than melting or softening temperature of the anti-crushing ingredient or the pH dependent ingredient used in the pharmaceutical composition. In thermal process, heat is employed to attain anticrushing property of the composition while in non-thermal process, heat is employed as per need of manufacturing process to evaporate binder or coating solvent during manufacturing process.

In pharmaceutical industry, thermal process is well known for making innovative pharmaceutical composition from last few decades. Examples of thermal process include hot melt extrusion (HME), melt granulation, curing of pharmaceutical composition at melting temperature, exposing of the composition to a heat during shaping into a dosage unit, etc.

In certain embodiment, a pharmaceutical abuse deterrent composition prepared by thermal process comprising step of exposing the pharmaceutical composition to the temperature which is at least melting or softening temperature of the anti-crushing ingredient or the pH dependent ingredient used in the pharmaceutical composition.

In certain embodiment, a pharmaceutical abuse deterrent composition prepared by thermal process comprising step of exposing mixture of the drug substance with at least one of the pH dependent ingredient and anti-crushing ingredient to the temperature which is at least melting or softening temperature of the anti-crushing ingredient or the pH dependent ingredient used in the pharmaceutical composition. The mixture further comprising at least one ingredient of pH independent ingredient, ion-exchange resin, water insoluble ingredient, gelling ingredient, diluent, glidant, surfactant, stabilizer, binder, lubricant, disintegrant, plasticizer, anti-tacking agent, surfactant, and stabilizer.

In certain embodiment, the pharmaceutical composition manufactured by thermal process exhibit breaking strength at least sufficient to resist pulverisation of the dosage form and thereby not more than about 35% pulverized particles of the pharmaceutical composition pass through #200 sieve (75 microns) when subjected to the coffee grinder for about 1 minutes.

In certain embodiment, each of the first strength and the second strength have resistance to alcohol dose dumping. Alcohol dose dumping resistance of the first strength and the second strength can be achieved by applying coat surrounding the particulates of the drug substance or the composition, wherein the coat comprising at least one ingredient which is insoluble in alcohol or 40% w/w alcohol in water but substantially a soluble in water.

The terms “resistance to alcohol dose dumping” are used to refer to two or more dosage units (e.g., any form(s) of tablets or capsules) that at least fulfil the condition that in vitro dissolution, characterized by the percentage of drug substance released at, e.g., 30 minutes or 60 minutes or 120 minutes of dissolution, when measured at 37°±1° C. in 900 ml 0.01N HCl comprising 40% ethanol in a USP Apparatus 2 (paddle) or USP apparatus 1 at 50 rpm and optionally with sinker, deviates no more than 35% from the corresponding in vitro dissolution measured at the same time point in the same apparatus at the same speed in 900 ml 0.01N HCl without ethanol at 37°±1° C. Such resistance to alcohol dose dumping deters the abuse of the dosage form.

### Drug Substance

In present invention, the terms “drug”, “active substance”, “active ingredient”, “active agent”, “drug substance”, “pharmacologically active agent”, “physiologically active agent”, “drug substance in physiologically active form” or “pharmaceutically acceptable derivatives of the drug substance” are used interchangeably herein to refer to a chemical compound that induces a desired pharmacological or physiological effect or biological activity. The terms pharmaceutically acceptable derivatives of the drug substance mentioned herein, include, but not limited to, salts, ether, stereo-isomer solvates, polymorphs, hydrates, complexes with one or more molecules, complexes with one or more cationic or anionic ingredient, drug-ion exchange resin complex, prodrug, active metabolites, analogs, homologue, and the like. In some embodiment, a drug substance is present in base form. The term drug substance is also meant to encompass the use of all such possible forms as well as their racemic and resolved forms thereof, and all tautomers as well. The term “racemic” refers to a mixture of equal parts of enantiomers.

In preferred embodiment, the drug substance is present in form of a pharmaceutically acceptable derivatives, wherein the pharmaceutically acceptable derivatives is drug-ion exchange resin, or complexes with one or more cationic or anionic ingredient.

In some embodiment, the drug substance is present in powder form, in amorphous form, in crystalline form, in micronized form, in complexes with one or more molecules, in combination of two or more active substance or any combination of thereof.

In some embodiments, the active ingredient include, but are not limited to, analgesics, anti-inflammatory agents, anti-helminthics, anti-arrhythmic agents, anti-asthma agents, anti-bacterial agents, anti-viral agents, anti-coagulants, anti-dementia agents, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, immunosuppressants, antiprotozoal agents, anti-thyroid agents, anti-tussives, anxiolytics, sedatives, hypnotics, neuroleptics, neuroprotective agents, β-blockers, cardie inotropic agents, cell adhesion inhibitors, corticosteroids, cytokine receptor activity modulators, diuretics, anti-Parkinson's agents, gastro-intestinal agents, histamine H-receptor antagonists, keratolyses, lipid regulating agents, muscle relaxants, nitrates and other anti-anginal agents, non-steroid anti-asthma agents, nutritional agents, opioid analgesics, sex hormones, stimulants and anti-erectile dysfunction agents; and salts, esters, and mixtures thereof.

In preferred embodiments, a drug substance is associated with abuse syndromes and the drug substance may, thus for example, be selected from opioids, opiates, CNS depressants or sedative, anxiolytics, narcotic, tranquilizers, barbiturates, hormones, CNS stimulants, cannabinoids, nicotine-like compounds, glutamate antagonists, N-methyl-D-aspartate (NMDA) antagonists or drugs that can cause psychological and/or physical dependence. Drugs that are preferred include those classified as Schedule I, II, III, IV and V drugs based upon the substance's medicinal value, harmfulness, and potential for abuse or addiction under the control substance act of United State.

The present invention is very particularly suitable for preventing overdose of active drug substances includes, but not limited to, are:

1-(1-Phenylcyclohexyl)pyrrolidine, 1-(2-Phenylethyl)-4-phenyl-4-acetoxypiperidine, 1-[1-(2-Thienyl)-cyclohexyl]piperidine, 1-[1-(2-Thienyl)cyclohexyl]pyrrolidine, 1-Methyl-4-phenyl-4-propionoxy-piperidine, 1-Phenylcyclohexylamine, 1-Piperidinocyclohexanecarbonitrile, 2,5-Dimethoxy-4-ethyl amphetamine, 2,5-Dimethoxyamphetamine, 2C-B-(4-bromo-2,5-dimethoxylpenethylamine), 2C-D (2,5-dimethoxy-4-methylphenethylamine), 2C-I (4-iodo-2,5-dimethoxy-phenethylamine), 2C-T-2 (2,5-dimethoxy-4-ethylthiophenethylamine), 2C-T-4 (2,5-dimethoxy-4-isopropyl thiophenethylamine), 2C-T-7 (2,5-dimethoxy-4-(n)-propylthiopenethylamine), 3,4-Methylene-dioxymethamphetamine, 3,4,5-Trimethoxyamphetamine, 3,4-Methylenedioxy amphetamine, 3,4-Methylenedioxy-N-ethylamphetamine, 3-Methylfentanyl, 3-Methylthiofentanyl, 4-Bromo-2,5-dimethoxyamphetamine, 4-Bromo-2,5-dimethoxyphenethylamine, 4-Methoxyamphetamine, 4-Methyl-2,5-dimethoxyamphetamine, 4-Methylaminorex (cis isomer), 5-MeO-DMT (5-Methoxy-N,N-diisopropyltryptamine), 5-MeO-DMT (5-Methoxy-N,N-dimethyltryptamine), 5-Methoxy-3,4-methylenedioxyamphetamine, Acetorphin, Acetorphine, Acetyl-alpha-methylfentanyl, Acetyl-alpha-methylfentanyl, Acetyldihydrocodeine, Acetylmethadol, Acetylmethadol, aniloridine, Alfentanil, Allobarbital, Allylprodin, Allylprodine, Alphacetylmethadol except levo-alphacetylmethadol, Alpha-ethyltryptamine, Alphameprodine, Alphamethadol, Alphamethadol, Alpha-Methylfentanyl, Alpha-Methylthiofentanyl, Alphaprodine, Alprazolam, Amfepramon, Amfetaminil, Amineptin, Aminorex, Amobarbital, amfepramone, Amphetamine, amphetaminil, Amylnitrit (all isomers of the amyl group), Anabolic steroids, Anileridine, apocodeine, Aprobarbital, Barbital, Barbituric acid derivative, BDB (3,4-methylenedioxyphenyl)-2-butanamine), Benzethidin, Benzethidine, Benzoylecgonine, Benzphetamine, Benzphetamine, Benzylmethylketon, Benzylmorphine, Betacetylmethadol, Beta-Hydroxy-3-methylfentanyl, Beta-Hydroxyfentanyl, Betameprodine, Betameprodine, Betamethadol, Betaprodine, Bezitramide, Boldenone, Brolamfetamin, Bromazepam, Brotizolam, Bufotenine, Buprenorphine, Butabarbital, Butalbital, Butobarbital, Butorphanol, BZP (A 2)(1-benzylpiperazin), Camazepam, Cannabis, Carfentanil, carfentanyl, Catha edulis, Cathine, Cathinone, Chloral betaine, Chloral hydrate, Chlordiazepoxide, Chlorhexadol, Chlorotestosterone (same as clostebol), Chlorphentermine, Clobazam, Clonazepam, Clonitazene, Clorazepate, Clortermine, Clostebol, Clotiazepam, Cloxazolam, Coca Leaves, Cocaine, Codeine, Codeine &isoquinoline alkaloid, Codeine methylbromide, Codeine-N-oxide, Codoxim, cyclorphan, Cyclobarbital (Hexemal NFN), cyclazocine, Cyprenorphine, Dehydrochlormethyltestosterone, Delorazepam, Desomorphine, Dexamfetamine, dexamphetamine, dexmethylphenidate, Dexfenfluramine, Dextromoramide, dextromethorphan, Dextropropoxyphene, dezocine, Diacetylmorphine, diamorphone, Diampromide, diapromide, Diazepam, Dichloralphenazone, Diethylpropion, Diethylthiambutene, Diethyltryptamine, Difenoxin, Dihydrocodeine, Dihydroetorphine, Dihydromorphine, Dihydrotestosterone, dimephetanol, Dimenoxadol, Dimepheptanol, Dimethylthiambutene, Dimethyltryptamine, Dioxaphetyl butyrate, Diphenoxylate, Dipipanone, Diprenorphine, Dronabinol, Drostanolone, Drotebanol, Ecgonine, eptazocine, Estazolam, Ethchlorvynol, Ethinamate, ethoheptazine, Ethyl loflazepate, Ethylestrenol, Ethylmethylthiambutene, Ethylmorphine, Eticyclidin, Etilamfetamine, Etonitazene, Etorphine, Etoxeridine, Etryptamine, Fencamfamin, Fenethylline, Fenetylline, Fenfluramine, Fenproporex, Fentanyl, Fludiazepam, Flunitrazepam, Fluoxymesterone, Flurazepam, Formebolone, Fungi and Spores of the species PsilocypeSemilanceata, Furethidine, Gammahydroxybutanic acid, Glutethimide, Halazepam, Hallucinogens, Haloxazolam, Heroine, Hydrocodone, Hydrocodone &isoquinoline alkaloid, Hydromorphinol, Hydromorphone, Hydroxypethidine, Ibogaine, Isobutylnitrit, Isomethadone, Ketamine, Ketazolam, Ketobemidone, lefetamine, Levamfetamine, levallorphan, Levo-alphacetylmethadol, Levo-methamphetamine, Levomethorphan, Levomoramide, Levophenacylmorphan, Levorphanol, lisdexamphetamine, lofentanil, loperamide, Loprazolam, Lorazepam, Lormetazepam, Lysergic acid, Lysergic acid amide, Lysergic acid diethylamide, Marijuana, Mazindol, MBDN(N-methyl-1-(3,4-methylenedioxyphenyl)-2-butanamine), mCPP (1-(3-chlorphenyl)piperazine), Mebutamate, Mecloqualone, Medazepam, Mefenorex, MeOPP (1-(4-methoxyphenyl)piperazine), Meperidine intermediate, Meprobamate, meptazinol, Mescaline, Mesocarb, Mesterolone, Metamfetamine, Metazocine, Methadone, Methadone intermediate, Methamphetamine, Methandienone, Methandranone, Methandriol, Methandrostenolone, Methaqualone, Methcathinone, Methenolone, Methohexital, Methyldesorphine, Methyl dihydromorphine, Methylphenidate, Methylphenobarbital (mephobarbital), Methyltestosterone, Methyprylone, Metopone, Mibolerone, Midazolam, Modafinil, Moramide-intermediate, Morpheridine, Morphine, morphine 3-glucuronide, morphine 6-glucuronide, Morphine methylbromide, Morphine methyl sulfonate, Morphine-N-oxide, Myrophine, N,N-Dimethylamphetamine, Nabilone, nalbuphine, Nalorphine, Nandrolone, narccine, N-Ethyl-1-phenylcyclohexylamine, N-Ethyl-3-piperidyl benzilate, N-Ethylamphetamine, N-Hydroxy-3,4-methylenedioxyamphetamine, Nicocodeine, Nicocodine, Nicodicodine, Nicomorphine, Nimetazepam, Nitrazepam, N-Methyl-3-piperidyl benzilate, Noracymethadol, Norcodeine, Nordiazepam, Norethandrolone, Norlevorphanol, Normethadone, Normorphine, Norpipanone, ohmefentanyl, Opium, Oxandrolone, Oxazepam, Oxazolam, oxycodeine, Oxycodone, Oxymesterone, Oxymetholone, Oxymorphone, papavereturn, Para-Fluorofentanyl, Parahexyl, Paraldehyde, pethidine, Pemoline, Pentazocine, Pentobarbital, Petrichloral, Peyote, Phenadoxone, Phenampromide, Phenazocine, Phencyclidine, Phendimetrazine, Phenmetrazine, Phenobarbital, Phenomorphan, Phenoperidine, Phentermine, Phenylacetone, Pholcodine, Piminodine, Pinazepam, Pipradrole, Piritramide, PMMA (paramethyxymethyl amphetamine), Prazepam, prodine, Proheptazine, promedol, Properidine, Propiram, propheptazine, propoxyphene, Psilocybine, Psilocyn, Pyrovalerone, Quazepam, Racemethorphane, Racemoramide, Racemorphane, Remifentanil, Salvia divinorum, Salvinorin A, Secobarbital, Secobarbital, Sibutramine, SPA, Stanolone, Stanozolol, Sufentanil, Sulfondiethylmethane, Sulfonethylmethane, Sulfonmethane, Talbutal, Tapentadol, Temazepam, Tenamfetamin, Testolactone, Testosterone, Tetrahydrocannabinols, Tetrazepam, TFMPP (1-(3-triflourmethylphenyl)piperazine), Thebacon, Thebaine, Thiamylal, Thiofentanyl, Thiopental, Tiletamine, Tramadol, Zolazepam, Zolazepam, Tilidine, Trenbolone, Triazolam, Trimeperidine, Vinbarbital, Zaleplon, Zipeprol, Zolpidem, Zopiclon.

In one embodiment of present invention, the drug substance may also include a new chemical entity for which the amount of information is limited. In such cases, the dosage form regimen needs to evaluate based on preclinical and clinical trials.

The above-mentioned drug substance may also be in the form of pharmaceutically acceptable salts, uncharged or charged molecules, molecular complexes, solvates or anhydrates thereof, and, if relevant, isomers, enantiomers, racemic mixtures, and mixture thereof.

In some embodiments, a dosage unit of the pharmaceutical composition contains an effective dose of drug to provide a therapeutic effect.

### Acid Neutralising Ingredient

In the present invention, the term “acid neutralising ingredient” may be used to refer to an excipient that acts to increase the pH of, e.g., the stomach fluid (e.g., roughly pH 1.2-4.5) or the aqueous fluid (e.g., 0.01N HCl with pH about 2.0) to a pH greater than about 5.5. For example, acid neutralising ingredient may refer to substances that are capable of increasing the pH to greater than 4.5, greater than 5.5, greater than 6.5, etc. It also refers to basic substances and substances that can convert an acidic environment to a less acidic or a basic environment. Typically, these agents, when present in a sufficient amount, are able to elevate the pH of the stomach fluid or the aqueous fluid to greater than about 4.5 or 5.5 and thereby prevent, reduce, or inhibit solubilisation of pH dependent ingredient.

In some embodiment, antacid may be used as acid neutralising ingredient.

In some embodiments, the acid neutralising ingredient is present in an amount of about 5 wt % to about 95 wt %; about 15 wt % to about 85 wt %; about 20 wt % to about 80 wt % or about 25 wt % to about 75 wt % of total weight of dosage form.

Suitable acid neutralising ingredient includes, but are not to be limited, magnesium oxide, meglumine, sodium oxide, sodium hydroxide, sodium bicarbonate, sodium potassium tartrate, bismuth aluminate, bismuth carbonate, bismuth subcarbonate, bismuth subgallate, bismuth subnitrate, potassium citrate, sodium citrate, sodium carbonate, potassium bicarbonate, potassium carbonate, calcium carbonate, calcium phosphate, dibasic calcium phosphate, dihydroxyaluminumaminoacetate, dihydroxyaluminum sodium carbonate, glycine, magnesium glycinate, magnesium hydroxide, magnesium carbonate, sodium borate, aluminum oxide, aluminum hydroxide, ammonium carbonate, monoethanolamine, diethanolamine, triethanolamine, potassium hydroxide, calcium hydroxide, sodium phosphate dibasic, trolamine, sodium potassium tartrate, tribasic sodium phosphate, tricalcium phosphate and like. The acid neutralising ingredient also include agents that inhibit acid secretion in stomach such as, but not to be limited, histamines receptor antagonists and proton pump inhibitors.

### pH Dependent Ingredient

In the present invention, the term “pH dependent ingredient” is used to refer an ingredient whose solubility vary according to the pH of the surrounding environment. The surrounding environment may comprise any type of liquid medium, such as gastrointestinal fluid, water, acid solution, alkaline solution, dissolution media, and like.

In some embodiments, pH dependent ingredient is present in an amount of about 0.5 wt % to about 75 wt %; about 1 wt % to about 55 wt %; about 2 wt % to about 35 wt % or about 3 wt % to about 25 wt % of total weight of dosage form.

In some embodiment, pH dependent ingredient include, but are not to be limited, calcium carbonate, chitin, chitosan, di and tribasic calcium phosphate, magnesium hydroxide, casein, polyvinylacetal diethylamino acetate (AEA), polymethacrylate or methacrylic acid and its derivative such as copolymer of dimethylaminoethyl methacrylate, butyl methacrylate, methyl methacrylate, copolymer of diethylaminoethyl methacrylate and methyl methacrylate, for example, Eudragit EPO, Eudragit E100, Eudragit 12.5, Kollicoat smartseal and like.

According to the present invention, pH dependent ingredient also includes those ingredient, other than mentioned in the present invention, whose characteristic and particularly solubility vary according to the pH of the physiological condition of the gastrointestinal fluid or the aqueous fluid.

### Aversion Agent

The term “aversion agent” is used herein to refer agent or ingredient that discourages the intention of incorrect administration of dosage form by producing aversive effect. Aversive effect means effect produced by the aversion agent that discourages incorrect administration of dosage form for obtaining intoxication. Aversive effect produced by the aversion agent include decrease or block the action of drug substance or produce feeling of dislike, feeling of uncomfortable or any unpleasant effect.

According to invention, the aversion agent suitable according to present invention include, but not to be limited, antagonist agent, emetic agent, flushing agent, irritating agent, burning agent and like. However, the aversion agent as describe here are for information, which not limit the scope of invention, i.e. agent other than described here may also be used as the aversion agent, for instance, agent which act according to definition of the aversion agent as described above.

In some embodiment, the aversion agent like antagonist agent, emetic agent or flushing agent are added in physiologically active form or in pharmacologically active form and in pharmaceutically acceptable form in dosage form including, but not limited to, free base, free acid, salts, solvates, hydrates, complexes with one or more molecules, prodrugs, active metabolites, and analogs. Free base or free acid forms of the aversion agent have pH dependent solubility in aqueous environment.

In preferred embodiment, dosage form may comprise at least one aversion agent or combination of two or more aversion agent of same category or of different category.

In some embodiment, antagonist agent can be used as the aversion agent to antagonize the effect of drug substance upon incorrect administration of dosage form. Selection of the antagonist agent is based on drug substance used in dosage form and it may vary according to therapeutic classes of drug substance. Antagonists useful in the present invention include, but not limited to, antagonists for opioids, non-opioid, central nervous system (CNS) depressants, stimulants, tranquilizers, barbiturates, hormones, cannabinoids, nicotine-like compounds, cold and cough drugs such as pseudoephedrine, glutamate antagonists, N-methyl-D-aspartate (NMDA) antagonists, etc. Suitable antagonist agents include, but are not limited to, naloxone, naltrexone, nalmefene, nalide, nalmexone, nalorphine, naluphine, haloperidol, promethazine, fluphenazine, perphenazine, levomepromazine, cyclazocine, thioridazine, perazine, chlorpromazine, chlorprothixine, zuclopentixol, flupentixol, prothipendyl, zotepine, benperidol, pipamperone, melperone, bromperidol and like.

In some embodiment, emetic agent can be used as the aversion agent. Emetics agent are agents which produce vomiting or emesis, which may be used in accordance with the invention as the aversion agent. Local or gastric emetics are the more rapid in their action, producing emesis in from two to five minutes. The systemic emetics must be absorbed and pass to the medulla before they produce vomiting, consequently requiring more time to exert their influence. Suitable emetic agents include, but not limited to, ipecacuanha, apomorphine, tartar emetic, zinc sulphate, copper sulphate, sodium chloride, mustard, ammonium carbonate and like.

In some embodiment, flushing agent can be used as the aversion agent in dosage form. For example, niacin, if taken in excessive dose, it produces warmth, flushing, and other uncomfortable symptoms.

In some embodiment, capsaicin can be used as irritating or burning agent in dosage form as the aversion agent. Capsaicin, if taken in excessive amount, it produces burning like feeling inside the stomach.

### Other Ingredients

In preferred embodiment, each of the first strength and the second strength individually further comprising at least one ingredient of pH independent ingredient, water insoluble ingredient, gelling ingredient, anti-crushing ingredient, diluent, glidant, surfactant, stabilizer, binder, lubricant, disintegrant, plasticizer and anti-tacking agent, in amount of not less than about 0.25% w/w and not more than about 90% w/w of the dosage unit.

The “pH independent ingredient” refers to a component whose characteristics do not generally vary according to the pH of the physiological condition of the gastrointestinal fluid. The pH independent ingredient is water soluble ingredient. Suitable water-soluble ingredient includes, but are not limited to, polymer, sugar, salts, salts of organic acid, acid and polysaccharide. Water soluble polymer include, but are not to be limited, cellulose derivatives include, but are not limited to be, methyl cellulose, hydroxypropyl methylcellulose, hydroxyethyl cellulose, hydroxypropyl cellulose, hydroxyethyl methylcellulose, ethylhydroxyethylcellulose, ethylmethylcellulose, hydroxymethylcellulose, hydroxymethylpropylcellulose, sodium carboxymethylcellulose, polyacrylamide derivatives, methacrylic acid derivatives, vinyl pyrrolidone polymers such as polyvinylpyrrolidone, starch derivative, polyalkylene oxide and copolymer thereof, alkylene oxide homopolymers, gums of plant, animal, mineral or synthetic origin, polyacrylic acid and copolymer thereof, polyvinyl alcohols, polyethylene glycol, poloxamer; and mixtures thereof. Preferred sugars include dextrose, glucose, arabinose, ribose, arabinose, xylose, lyxose, xylol, allose, altrose, inositol, glucose, sorbitol, mannose, gulose, glycerol, idose, galactose, talose, trehalose, mannitol, erythritol, ribitol, xylitol, maltitol, isomalt, lactitol, sucrose, raffinose, maltose, fructose, lactose, dextrin, dextran, amylase and xylan. Water soluble salts include sodium chloride, potassium chloride, calcium chloride or magnesium chloride, lithium chloride, lithium, sodium or potassium hydrogen phosphate, lithium, sodium or potassium dihydrogen phosphate, salts of organic acids such as sodium or potassium acetate, magnesium succinate, sodium benzoate, sodium citrate or sodium ascorbate. Preferred acids include ascorbic acid, 2-benzene carboxylic acid, benzoic acid, fumaric acid, citric acid, maleic acid, serbacic acid, sorbic acid, edipic acid, edetic acid, glutamic acid, toluene sulfonic acid, water-soluble amino acids such as glycine, leucine, alanine, or methionine and tartaric acid; and like. Polysaccharides are polymeric carbohydrate molecules composed of long chains of monosaccharide units bound together by glycosidic linkages and on hydrolysis give the constituent monosaccharides or oligosaccharides. They range in structure from linear to highly branched. Examples include storage polysaccharides such as starch and glycogen, and structural polysaccharides such as cellulose and chitin.

The term “water insoluble ingredient” refers to a component which is insoluble in water. Suitable water insoluble ingredient includes natural, synthetic or semi synthetic ingredient. Natural, synthetic or semi synthetic water insoluble ingredient include, but are not to be limited, cellulose derivatives include cellulose acetate, cellulose acetate butyrate, cellulose triacetate, microcrystalline cellulose, ethyl cellulose, glycerol palmitostearate, wax include microcrystalline wax, beeswax, glycowax, castor wax, carnauba wax, glycerol monostearate, oil include hydrogenated vegetable oil, hydrogenated castor oil, vegetable oil, stearyl alcohol, acetylated hydrogenated soybean oil glycerides, castor oil, glycerol behenic acid ester, glyceryl monooleate, glyceryl monostearate, propylene glycol monostearate, cetyl alcohol, natural and synthetic glycerides, fatty acids, fatty alcohol, lipid, methacrylic acid derivatives such polymethaacrylate and its copolymer (such as eudragit polymer); polyvinyl acetate, copolymers of vinyl pyrrolidone and vinyl acetate; vinyl acetate and copolymer thereof, ethyl vinyl acetate, modified starch like pregelatinised starch, polylactic acid or polyglycolic acid and copolymers thereof, methacrylates, cocoa butter, macrogol Stearate, diethylene glycol monostearate, polyoxyethylene 50 stearate, and mixtures thereof.

The term “gelling ingredient” as used herein refers to an ingredient that increase the viscosity of aqueous or non-aqueous media. Suitable gelling ingredient is at least one of natural, semi-synthetic or synthetic ingredient which includes at least one of gum, polysaccharide, water soluble polymer, water soluble protein, and starch. Suitable gelling ingredient includes, but not limited to, xanthan gum, acacia gum, diutan gum, tragacanth, gellan gum, guar gum, fenugreek gum, locust bean gum, pullulan, welan gum, starch or its derivative, celluloseor its derivative (such as hydroxyethyl cellulose, hydroxypropylmethyl cellulose), polyalkylene oxide and its co-polymer such as polyethylene oxide and copolymer of ethylene oxide—propylene oxide, ploycarboxylic acid such as polyacrylic acid, polypeptide such as gelatin, albumin, polylysine, soy protein, polyolefinic alcohol (such as polyvinyl alcohol), or a polyvinyl lactam such as, e.g., polyvinylpyrrolidone, polyvinyl caprolactam, alginic acid and its derivative, methacrylic acid and its copolymer, polyacrylic acid and copolymer thereof, and like. According to invention, more preferable gelling ingredient has cloud point greater than about 85° C. Gelling ingredient having cloud point greater than about 85° C. resist separation of the drug substance from a dosage unit by hot water having temperature greater than about 90° C. and thereby create a complexity in efforts of getting pure drug substance from the dosage unit.

The anti-crushing ingredient include thermoplastic material such as polyalkylene oxide or its copolymer, polyvinyl acetate, polysaccharide such as starch or its derivative, cellulose or its derivatives or glycogen, or mixture thereof.

Suitable disintegrant includes, but are not limited to, cellulose derivatives, including microcrystalline cellulose, low-substituted hydroxypropyl cellulose, ion-exchange resin, starch and its derivative, croscarmellose sodium, alginic acid, insoluble polyvinylpyrrolidone, and like.

Suitable plasticizers includes, but are not limited to, triacetin, triethyl acetate, acetylated monoglyceride, olive oil, acetyl tributyl citrate, acetyl triethyl citrate, glycerin, sorbitol, polyethylene glycol, polypropyleneglycol and like.

Suitable diluents includes, but are not limited to, sucrose, sorbitol, mannitol, various grades of lactose, various grades of microcrystalline cellulose, dextrins, maltodextrins, starches or modified starches, sodium phosphate, calcium phosphate, calcium carbonate, and like.

Suitable glidants and lubricants may be incorporated such as stearic acid, metallic stearates, talc, waxes, and glycerides with high melting temperatures, colloidal silica, sodium stearyl fumarate, polyethyleneglycols, and alkyl sulphates.

Suitable surfactants include, but are not limited to, non-ionic surfactants, anionic surfactants, and cationic surfactants. Example of surfactant include, but are not limited to, ammonium lauryl sulfate, sodium lauryl sulfate, docusate (dioctyl sodium sulfosuccinate), perfluorooctanesulfonate, perfluorobutanesulfonate, alkyl-aryl ether phosphates, alkyl ether phosphates, sodium stearate, sodium lauroylsarcosinate, perfluorononanoate, perfluorooctanoate, cetrimonium bromide, cetylpyridinium chloride, benzalkonium chloride, benzethonium chloride, dimethyldioctadecyl ammonium chloride, dioctadecyldimethylammonium bromide, phospholipids, phosphatidyl serine, phosphatidylethanolamine, phosphatidylcholine, sphingomyelins, poloxamer, fatty acid ester of glycerol, fatty acid ester of sorbitol such as tween and sorbitan, and like.

Suitable anti-tacking agent is selected from the group consisting of, but are not limited to, stearates, stearic acid, vegetable oil, waxes, a blend of magnesium stearate and sodium lauryl sulfate, boric acid, surfactants, sodium benzoate, sodium acetate, sodium chloride, DL-Leucine, polyethylene glycol, sodium oleate, sodium lauryl sulfate, magnesium lauryl sulfate, talc, corn starch, amorphous silicon dioxide, syloid, metallic stearates, Vitamin E, Vitamin E TPGS, silica and combinations thereof.

Suitable binder include, but are not to be limited, cellulose derivatives include, but are not limited to be, methyl cellulose, hydroxypropyl methylcellulose, hydroxyethyl cellulose, hydroxypropyl cellulose, hydroxyethyl methylcellulose, ethylhydroxyethylcellulose, ethylmethylcellulose, hydroxymethylcellulose, hydroxymethylpropylcellulose, sodium carboxymethylcellulose, polyacrylamide derivatives, methacrylic acid derivatives, vinyl pyrrolidone polymers such as polyvinylpyrrolidone, starch derivative, polyalkylene oxide and copolymer thereof, alkylene oxide homopolymers, gums of plant, animal, mineral or synthetic origin, polyacrylic acid and copolymer thereof, polyvinyl alcohols, polyethylene glycol, poloxamer, and mixtures thereof.

In certain embodiment, some ingredient has more than one functionality in the composition. For example, polyethylene oxide can be used as anti-crushing ingredient as well as gelling ingredient having cloud point greater than about 80° C.

In preferred embodiment, the pharmaceutical composition further comprising any functional ingredient mentioned in the Handbook of Pharmaceutical excipients, 7^{th} edition. The term “functional ingredient” refer to an ingredient that add some functional characteristic, as mentioned in application part (use of ingredient) of each ingredient in the Handbook of Pharmaceutical excipients, in to the composition.

In certain embodiment, a pharmaceutical abuse deterrent composition can be formulated in the form of a sprinkle formulation. Sprinkle formulation is useful for children and other patients, who have difficulty swallowing the conventional solid dosage forms. The sprinkle formulation may be administered without the need to take it with water. The term “sprinkle formulation” includes any formulation that is suitable for oral administration, wherein the formulation is sprinkled upon any consumable item. According to invention, “Sprinkle formulation” is coated particulates of the pharmaceutical abuse deterrent composition and is < about 2.8 mm in size and can be administered orally with food with or without chewing.

### EXAMPLES

Certain aspects of the present invention may be better understood as illustrated by the following examples, which are meant by way of illustration and not limitation.

### Example 1

| | | | Quantity (% w/w) | |
|---|---|---|---|---|
| Sr. | | | 60 mg | 15 mg |
| No. | Ingredient | Functionality of Each Ingredient | Strength | Strength |

Crush-Resistant Mini-Tablet

| | | | |
|---|---|---|---|
| 1 | Drug Substance | Drug Substance | 10.00 |
| 2 | Polyethylene Oxide | Matrixing agent, Anti-Crushing | 73.38 |
| | (PEO) | Ingredient & Gelling Ingredient | |
| 3 | Magnesium Stearate | Lubricant | 0.74 |

Functional Coat

| | | | |
|---|---|---|---|
| 4 | Cellulose Acetate | Alcohol Insoluble Ingredient & Water | 3.12 |
| | | Insoluble Ingredient | |
| 5 | Poloxamer | Water Soluble Ingredient | 0.88 |

Outer Coat

| | | | |
|---|---|---|---|
| 6 | Eudragit EPO | pH Dependent Ingredient | 8.00 |
| 7 | Magnesium Stearate | Anti-tacking Ingredient | 3.88 |

Acid Neutralising Composition

| | | | Quantity (mg) | |
|---|---|---|---|---|
| Sr. | | Functionality of Each | 60 mg | 15 mg |
| No. | Ingredient | Ingredient | Strength | Strength |
| 1 | Sodium Bicarbonate | Acid Neutralising Ingredient | 160 | 40 |
| 2 | Magnesium Oxide | Acid Neutralising Ingredient | 32 | 8 |

Manufacturing Procedure:
1. Preparation of Mini-Tablets: #30mesh passed drug, PEO and magnesium stearate were mixed and compressed to form mini-tablets.
2. Functional Coat: The coating was performed on the step (1) mini-tablets using non-aqueous coating solution comprising mixture of cellulose acetate and poloxamer.
3. Preparation of Crush Resistant Mini-Tablets: Step (2) coated mini-tablets were cured at 60-70° C. bed temperature for period of 1 hour.
4. Outer Coat Surrounding the Crush Resistant Mini-Tablets: The coating was performed on the step (3) Crush Resistant Mini-Tablets using non-aqueous coating solution comprising mixture of Eudragit E-100 and magnesium Stearate.
5. Preparation of 60 mg Strength (first strength): 600 mg step (4) coated mini-tablets, 160 mg sodium bicarbonate and 32 mg magnesium oxide was filled in suitable size capsule.
6. Preparation of 15 mg Strength (second strength): 150 mg step (4) coated mini-tablets, 40 mg sodium bicarbonate and 8 mg magnesium oxide was filled in suitable size capsule.
7. In-vitro Dissolution:
   In-vitro dissolution testing of the first strength, the second strength, multitude of the 5 dosage units of the first strength and multitude of the 5 dosage units of the second strength was performed at 37°±1° C. in 900 ml 0.01N HCl in a USP Apparatus 2 (paddle) at 50 rpm for 1, 2, 4, 6, 8 and 10 hrs time point in the following sequence:
   1. First strength—60 mg (single dosage unit): 1 capsule/dissolution vessel
   2. Second Strength—15 mg (single dosage unit): 1 capsule/dissolution vessel
   3. Multitude of 5 dosage units of the First Strength (60 mg): 5 capsules of 60 mg/dissolution vessel—the overall content of the drug substance in the multitude of 5 capsules of the first strength is 300 mg/dissolution vessel.
   4. Multitude of 5 dosage units of the Second Strength (15 mg): 5 capsules of 15 mg/dissolution vessel—the overall content of the drug substance in the multitude of 5 capsule of the second strength is 75 mg/dissolution vessel.

| | % Drug Release (Mean) | | | |
|---|---|---|---|---|
| | | | Multitude of 5 Dosage unit | |
| | | | (3 or more dosage units) | |
| | | | | Second |
| | | | | Strength: |
| | | | | Multitude |
| | | | | of 5 |
| | Single dosage unit | | First Strength: | dosage |
| | | Second | Multitude of 5 | units of the |
| | | Strength: | dosage units of | Second |
| | First Strength: | 1 capsule per | the First Strength | Strength per |
| Time | 1 capsule per | dissolution | per dissolution | dissolution |
| (hours) | dissolution vessel | vessel | vessel | vessel |
| 1 | 0 | 0 | 0 | 0 |
| 2 | 23 | 21 | 1 | 19 |
| 4 | 48 | 45 | 3 | 41 |
| 6 | 72 | 69 | 8 | 62 |
| 8 | 91 | 90 | 16 | 86 |
| 10 | 98 | 96 | 28 | 91 |

| | | |
|---|---|---|
| F2 Value | 81 | 16 |
| | (F2 Value: > 50) | (F2 Value: < 50) |
| Remarks | The particulates in a single dosage | The particulates in a multitude of 3 or |
| | unit of each strength has similar | more dosage units of each strength has |
| | release profile of the drug substance | different release profile of the drug |
| | relative to the other strength because | substance relative to the other strength |
| | F2 Value is > 50 | because F2 Value is < 50 |

### Example 2

| | | | Quantity (mg) | | |
|---|---|---|---|---|---|
| | | Functionality of Each | 30 mg | 20 mg | 10 mg |
| Sr. No. | Ingredient | Ingredient | Strength | Strength | Strength |

Composition for Particulates of Drug Substance
Crush Resistant Particulates

| | | | | | |
|---|---|---|---|---|---|
| 1 | Polyethylene Oxide | Gelling Ingredient & | 120.0 | 80 | 40 |
| | (#30 mesh passed and | Anti-crushing Ingredient | | | |
| | #60 Mesh Retained- | | | | |
| | 250 to 600 micron) | | | | |
| 2 | Hypromellose | pH independent | 3.0 | 2.0 | 1.0 |
| | | ingredient | | | |
| 3 | Ethyl Cellulose | Water Insoluble | 27.0 | 18.0 | 9.0 |
| | | Ingredient | | | |

Coat of Drug Substance

| | | | | | |
|---|---|---|---|---|---|
| 4 | Model Drug (Water | Drug Substance | 30.0 | 20.0 | 10.0 |
| | Soluble Drug) | | | | |
| 5 | Hypromellose | Binder | 15.0 | 10.0 | 5.0 |

Outer Coat of pH Dependent Ingredient

| | | | | | |
|---|---|---|---|---|---|
| 6 | Eudragit E-100 | pH Dependent | 127.5 | 85.0 | 42.5 |
| | | Ingredient | | | |
| 7 | Magnesium Stearate | Anti-tacking Ingredient | 52.5 | 35.0 | 17.5 |

| | | | |
|---|---|---|---|
| Total Weight of Particulates of Drug Substance | 375.0 | 250.0 | 125.0 |

Composition for Acid Neutralisation

| | | | | | |
|---|---|---|---|---|---|
| 8 | Sodium Bicarbonate | Acid Neutralising | 150.0 | 100.0 | 50.0 |
| | | Ingredient | | | |
| 9 | Magnesium Oxide | Acid Neutralising | 24.0 | 16.0 | 8.0 |
| | | Ingredient | | | |

| | | | |
|---|---|---|---|
| Total Weight of the Composition | 510.0 | 340.0 | 170.0 |

### Manufacturing Procedure:

1. Preparation of Crush Resistant Particulates: #30mesh passed and #60mesh retained polyethylene oxide was transferred in bottom spray bowl of Fluid Bed Coater and coating was performed on the polyethylene oxide using non-aqueous coating solution comprising mixture of ethyl cellulose and hypromellose. The coated particulates was passed through appropriate screen to get uniform size particulates.
2. Drug Coat surrounding the Crush Resistant Particulates: The coating was performed on the step (1) crush resistant particulates using aqueous coating solution comprising mixture of water soluble model drug and hypromellose.
3. Outer Coat of pH Dependent Ingredient surrounding the Drug Coated Particulates: The coating was performed on the step (2) drug coated particulates using non-aqueous coating solution comprising mixture of Eudragit E-100 and magnesium Stearate.
4. Preparation of 30 mg Strength: 375 mg step (3) coated particulate, 150 mg sodium bicarbonate and 24 mg magnesium oxide was filled in suitable size capsule.
5. Preparation of 20 mg Strength: 250 mg step (3) coated particulate, 100 mg sodium bicarbonate and 16 mg magnesium oxide was filled in suitable size capsule.
6. Preparation of 10 mg Strength: 125 mg step (3) coated particulate, 50 mg sodium bicarbonate and 8 mg magnesium oxide was filled in suitable size capsule.
   Note: Supratherapeutic Dose: 3 capsules of the first strength (30 mg)=90 mg

### Example 3

In-vitro dissolution testing of the first strength, the second strength and multitude of the second strength manufactured as per example 2 was performed at 37°±1° C. in 900 ml 0.01N HCl in a USP Apparatus 2 (paddle) at 50 rpm for 30 minutes time point in the following sequence:
1. First strength—30 mg: 1 capsule/dissolution vessel
2. Second Strength—10 mg: 1 capsule/dissolution vessel
3. Multitude of the second strength—10 mg: 6 capsules/dissolution vessel—the overall content of the drug substance originally contained in the multitude of 6 capsules of second strength is 60 mg which is greater than the content of the drug substance originally contained in the first strength (30 mg) but lower than the supratherapeutic dose of the drug substance which is 90 mg.
4. Multitude of the second strength—10 mg: 12 capsules/dissolution vessel—the overall content of the drug substance originally contained in the multitude of 12 capsules of second strength is 120 mg which is greater than supratherapeutic dose of the drug substance which is 90 mg.

| | | 30 | |
|---|---|---|---|
| Time (Minutes) | | Minutes | Remarks |
| Percentage | First Strength-30 mg | 91 | — |
| Drug Release | (1 capsule per dissolution | | |
| | vessel = 30 mg/Vessel) | | |
| | Second Strength-10 mg | 90 | Difference between the drug substance |
| | (1 capsule per dissolution | | release from the first strength and the |
| | vessel = 10 mg/Vessel) | | drug substance release from the second |
| | | | strength is 1 (less than about 25) |
| | Multitude of the second | 79 | Difference between the drug substance |
| | strength-10 mg | | release from the first strength and the |
| | (6 capsules per dissolution | | drug substance release from a multitude |
| | vessel = 60 mg/Vessel) | | of the second strength is 12 (less than |
| | Supratherapeutic Dose: 90 mg | | about 25), wherein the overall content of |
| | | | the drug substance originally contained |
| | | | in the multitude of the second strength is |
| | | | 60 mg which is greater than the content |
| | | | of the drug substance originally |
| | | | contained in the first strength (30 mg) but |
| | | | lower than the supratherapeutic dose of |
| | | | the drug substance which is 90 mg |
| | Multitude of the second | 19 | Difference between the drug substance |
| | strength-10 mg | | release from the first strength and the |
| | (12 capsules per dissolution | | drug substance release from a multitude |
| | vessel = 120 mg/Vessel) | | of the second strength is 72 (more than |
| | Supratherapeutic Dose: 90 mg | | about 25), wherein the overall content of |
| | | | the drug substance originally contained |
| | | | in the multitude of the second strength is |
| | | | 120 mg which is greater than the |
| | | | supratherapeutic dose of the drug |
| | | | substance which is 90 mg |

### Example 4

In-vitro dissolution testing of the first strength, the second strength and multitude of the second strength manufactured as per example 2 was performed at 37°±1° C. in 900 ml 0.01N HCl in a USP Apparatus 2 (paddle) at 50 rpm for 30 minutes time point in the following sequence:
1. First strength—30 mg: 1 capsule/dissolution vessel
2. Second Strength—20 mg: 1 capsule/dissolution vessel
3. Multitude of the second strength—20 mg: 3 capsules/dissolution vessel—the overall content of the drug substance originally contained in the multitude of 3 capsules of second strength is 60 mg which is greater than the content of the drug substance originally contained in the first strength (30 mg) but lower than the supratherapeutic dose of the drug substance which is 90 mg
4. Multitude of the second strength—20 mg: 6 capsules/dissolution vessel—the overall content of the drug substance originally contained in the multitude of 6 capsules of the second strength is 120 mg which is greater than supratherapeutic dose of the drug substance which is 90 mg

| | | 30 | |
|---|---|---|---|
| Time (Minutes) | | Minutes | Remarks |
| Percentage | First Strength-30 mg | 91 | — |
| Drug Release | (1 capsule per | | |
| | dissolution vessel = | | |
| | 30 mg/Vessel) | | |
| | Second Strength- | 87 | The second strength has released more than |
| | 20 mg | | about 70% wt. of the drug substance content |
| | (1 capsule per | | originally contained in the second strength |
| | dissolution vessel = | | |
| | 20 mg/Vessel) | | |
| | Multitude of the second | 75 | A multitude of the second strength has released |
| | strength-20 mg | | more than about 70% wt. of the overall content |
| | (3 capsules per | | of the drug substance originally contained in |
| | dissolution vessel = | | the multitude of the second strength, wherein |
| | 60 mg/Vessel) | | the overall content of the drug substance |
| | Supratherapeutic Dose: | | originally contained in the multitude of the |
| | 90 mg | | second strength is 60 mg which is greater than |
| | | | the content of the drug substance originally |
| | | | contained in the first strength (30 mg) but lower |
| | | | than the supratherapeutic dose of the drug |
| | | | substance which is 90 mg |
| | Multitude of the second | 25 | A multitude of the second strength has released |
| | strength-20 mg | | less than about 70% wt. of the overall content |
| | (6 capsules per | | of the drug substance originally contained in |
| | dissolution vessel = | | the multitude of the second strength, wherein |
| | 120 mg/Vessel) | | the overall content of the drug substance |
| | Supratherapeutic Dose: | | originally contained in the multitude of the |
| | 90 mg | | second strength is 120 mg which is greater than |
| | | | the supratherapeutic dose of the drug substance |
| | | | which is 90 mg. |

### Example 5

In-vitro dissolution testing of the first strength, the second strength and multitude of the second strength manufactured as per example 2 was performed at 37°±1° C. in 900 ml 0.01N HCl in a USP Apparatus 2 (paddle) at 50 rpm for 30 minutes time point in the following sequence:
1. First strength—30 mg: 1 capsule/dissolution vessel
2. Second Strength—10 mg: 1 capsule/dissolution vessel
3. First Strength (30 mg)+Second Strength (10 mg)−30 mg+10 mg: 1 capsule of 30 mg+1 capsule of 10 mg/dissolution vessel—the overall content of 1 capsule of second strength and 1 capsule of first strength is 40 mg which is greater than the content of the drug substance originally contained in the first strength (30 mg) but lower than the supratherapeutic dose of the drug substance which is 90 mg
4. First Strength (30 mg)+a multitude of Second Strength (3 Capsules)−30 mg+10 mg+10 mg+10 mg: 1 capsule of 30 mg+3 capsules of 10 mg/dissolution vessel—the overall content of 3 capsules of second strength and 1 capsule of first strength is 60 mg which is greater than the content of the drug substance originally contained in the first strength (30 mg) but lower than the supratherapeutic dose of the drug substance which is 90 mg
5. First Strength (30 mg)+a multitude of Second Strength (8 Capsules)−30 mg+10 mg+10 mg+10 mg+10 mg+10 mg+10 mg+10 mg+10 mg: 1 capsule of 30 mg+8 capsules of 10 mg/dissolution vessel—the overall content of 8 capsules of second strength and 1 capsule of first strength is 110 mg which is greater than the supratherapeutic dose of the drug substance which is 90 mg.

| | | 30 | |
|---|---|---|---|
| Time (Minutes) | | Minutes | Remarks |
| Percentage | First Strength-30 mg | 91 | — |
| Drug Release | (1 capsule per dissolution | | |
| | vessel = 40 mg/Vessel) | | |
| | Second Strength-10 mg | 90 | Released is more than about 70% wt. |
| | (1 capsule per dissolution vessel = | | of the drug substance content |
| | 10 mg/Vessel) | | originally present |
| | First Strength-30 mg + Second | 92 | Released is more than about 70% wt. |
| | Strength-10 mg | | of the drug substance content |
| | (1 capsule of 30 mg + 1 capsule of | | originally present (40 mg-1 capsule |
| | 10 mg/dissolution vessel = | | of 10 mg + 1 capsule of 30 mg)/Vessel |
| | 40 mg/Vessel) | | |
| | First Strength-30 mg) + a multitude | 75 | Released is more than about 70% wt. |
| | of 3 capsules of Second Strength- | | of the drug substance content |
| | 10 mg | | originally present (60 mg-3 capsules |
| | (1 capsule of 30 mg + 3 capsules of | | of 10 mg + 1 capsule of 30 mg)/Vessel |
| | 10 mg/dissolution vessel = | | |
| | 60 mg/Vessel) | | |
| | Supratherapeutic Dose: 90 mg | | |
| | First Strength-30 mg + a multitude | 28 | Released is less than about 70% wt. |
| | of 8 capsules of Second Strength- | | of the drug substance content |
| | 10 mg | | originally present (110 mg-8 |
| | (1 capsule of 30 mg + 8 capsules of | | capsules of 10 mg + 1 capsule of |
| | 10 mg/dissolution vessel = | | 30 mg)/Vessel |
| | 110 mg/Vessel) | | |
| | Supratherapeutic Dose: 90 mg | | |

### Example 6

5 grams of the drug substance particulates of example 2 were subjected to pulverisation using a coffee grinder for 1 minute and the resultant crushed drug substance particulates were sifted through #200 mesh sieve. The crushed drug substance particulates passed through #200 mesh sieve was less than 35% of initial weight of the drug substance particulate, i.e. 5 grams.

### Example 7

0.5 gram of the drug substance particulates of example 2 were subjected to pulverisation using a coffee grinder for 5 minutes. When the resultant crushed drug substance mixed with 5 ml purified water, it form high viscous mixture which is unsuitable for injection.

### Example 8

| | | | |
|---|---|---|---|
| | | Functionality of Each | Quantity |
| Sr. No. | Ingredient | Ingredient | (mg) |

Crush Resistant Particulates

| | | | |
|---|---|---|---|
| 1 | Polyethylene Oxide | Water soluble anti- | 80 |
| | (Particle Size: 430 mesh passed | crushing ingredient | |
| | and #60 Mesh Retained-250 to | | |
| | 600 micron) | | |

First Coat

| | | | |
|---|---|---|---|
| 2 | Povidone | pH independent ingredient | 2.0 |
| 3 | Ethyl Cellulose | Water Insoluble Ingredient | 18.0 |
| 4 | Ethanol | Non-Aqueous Solvent | q.s. |

Second Coat

| | | | |
|---|---|---|---|
| 5 | Model Drug (Water Soluble | Active Ingredient | 20.0 |
| | Drug) | | |
| 6 | Hypromellose | Non-Active Ingredient | 10.0 |

Outer Coat

| | | | |
|---|---|---|---|
| 7 | Ethyl Cellulose | Water Insoluble Ingredient | 85.0 |
| 8 | Hypromellose | pH independent ingredient | 35.0 |
| 9 | Isopropyl Alcohol | Non-Aqueous Solvent | q.s. |
| 10 | Dichloromethane | Non-Aqueous Solvent | q.s. |

| | |
|---|---|
| Total Weight of Particulates | 250.0 |

### Manufacturing Procedure:

1. Preparation of Crush Resistant Particulates: #30mesh passed and #60mesh retained polyethylene oxide was transferred in bottom spray bowl of Fluid Bed Coater.
2. First Non-aqueous Coat: The first non aqueous coating solution was prepared by dissolving ethyl cellulose and povidone in ethanol. The coating was performed on the polyethylene oxide in fluid bed coater using the non-aqueous coating solution. The coated particulates was then passed through appropriate screen to get uniform size particulates.
3. Second Coat: The coating was performed on the step (2) coated particulates using aqueous coating solution comprising mixture of water soluble model drug and hypromellose.
4. Outer Coat: A outer coating solution was prepared by dissolving mixture of ethyl cellulose and hypromellose in solvent comprising mixture of Isopropyl Alcohol and Dichloromethane. The coating was performed on the step (3) drug coated particulates using the outer coating solution.

### Example 9

Less than 10% w/w particulates were collected when 1 gram particulates of example 8 were sifted through #200mesh sieve.

### Example 10

5 grams of the drug substance particulates of example 8 were subjected to pulverisation using a coffee grinder for 1 minute and the resultant crushed drug substance particulates were sifted through #400 mesh sieve. The crushed drug substance particulates passed through #400 mesh sieve was less than 40% of initial weight of the drug substance particulate, i.e. 5 grams.

### Example 11

0.5 gram of the drug substance particulates of example 8 were subjected to pulverisation using a coffee grinder for 5 minutes. When the resultant crushed drug substance mixed with 5 ml purified water, it form highly viscous mixture which is unsuitable for injection.

We claim:

1. A pharmaceutical abuse deterrent composition containing particulates in which a release of a drug substance is controlled,
wherein the composition is constructed in more than one strength which comprises a first strength and a second strength, each of the first strength and the second strength individually contain an acid neutralising ingredient and the particulates of the drug substance, wherein
a. an amount of the particulates of the drug substance in the first strength is at least about 10% higher relative to the second strength, and
b. an amount of the acid neutralising ingredient in the second strength is at least about 5% lower relative to the first strength,
wherein the release of the drug substance in each particulate of each strength is controlled by a pH dependent ingredient depending on pH of surrounding environment which is raise or maintained by the acid neutralising ingredient, wherein
a. the pH dependent ingredient is present in the each particulate of drug substance as
1) matrixing agent in which the drug substance is embedded, or
2) an outer coating layer surrounding the particulate containing the drug substance, or
3) a combination of the step 1) and the step 2), and
b. the acid neutralising ingredient is present as separate particulates or coated on the particulates of drug substance,
wherein the particulates in a single dosage unit of each strength has similar release profile of the drug substance relative to the other strength but the particulates in a multitude of 3 or more dosage units of each strength has different release profile of the drug substance relative to the other strength when subjected to in-vitro dissolution testing.

2. A pharmaceutical abuse deterrent composition of claim 1, wherein the pH dependent ingredient is at least one of
a. polyvinylacetal diethylamino acetate,
b. copolymer of dimethylaminoethyl methacrylate, butyl methacrylate, methyl methacrylate, and
c. copolymer of diethylaminoethyl methacrylate and methyl methacrylate.

3. A pharmaceutical abuse deterrent composition of claim 1, wherein the acid neutralising ingredient is at least one of magnesium oxide, meglumine, sodium oxide, sodium hydroxide, sodium bicarbonate, sodium potassium tartrate, bismuth aluminate, bismuth carbonate, bismuth subcarbonate, bismuth subgallate, bismuth subnitrate, potassium citrate, sodium citrate, sodium carbonate, potassium bicarbonate, potassium carbonate, calcium carbonate, calcium phosphate, dibasic calcium phosphate, dihydroxyaluminumaminoacetate, dihydroxyaluminum sodium carbonate, glycine, magnesium glycinate, magnesium hydroxide, magnesium carbonate, sodium borate, aluminum oxide, aluminum hydroxide, ammonium carbonate, monoethanolamine, diethanolamine, triethanolamine, potassium hydroxide, calcium hydroxide, sodium phosphate dibasic, trolamine, sodium potassium tartrate, tribasic sodium phosphate and tricalcium phosphate.

4. A pharmaceutical abuse deterrent composition of claim 1, wherein the particulates of the drug substance have resistance to alcohol dose dumping.

5. A pharmaceutical abuse deterrent composition of claim 1, wherein each of the first strength and the second strength individually further containing particulates of an aversion agent, wherein the aversion agent is not released in effective amount to produce its intended aversive effect unless the composition is administered after physical alteration but is released in effective amount to produce its intended aversive effect when the composition is administered after the physical alteration.

6. A pharmaceutical abuse deterrent composition of claim 5, wherein the aversion agent is present as coated particulates, wherein each coated particulate comprising
1) a particulate comprising the aversion agent, wherein the aversion agent is present
a) as a particle or
b) within matrix of at least one ingredient of pH dependent ingredient, pH independent ingredient and water insoluble ingredient or
c) as a coat surrounding at least one of pH dependent ingredient, pH independent ingredient and water insoluble ingredient, and
2) water insoluble coat surrounding the particulate.

7. A pharmaceutical abuse deterrent composition of claim 1, wherein the particulate of the drug substance further comprising a functional ingredient to attain modified release profile of the drug substance, wherein the functional ingredient is at least one of pH independent ingredient and water insoluble ingredient.

8. A pharmaceutical abuse deterrent composition containing particulates in which a release of a drug substance is controlled,
wherein the composition is constructed in more than one strength which comprises a first strength having a first acid neutralisation capacity and a second strength having a second acid neutralisation capacity, each of the first strength and the second strength individually contain an acid neutralising ingredient and the particulates of the drug substance, wherein
a. an amount of the particulates of the drug substance in the first strength is at least about 10% higher relative to the second strength, and
b. the second acid neutralisation capacity of the second strength is lower than the first acid neutralisation capacity of the first strength,
wherein the first strength, the second strength and the multitude of the second strength when subjected to in-vitro dissolution testing for at least one corresponding time point:
a) the second strength has released more than about 70% wt. of the drug substance content originally contained in the second strength,
b) a multitude of the second strength has released more than about 70% wt. of the overall content of the drug substance originally contained in the multitude of the second strength, wherein the overall content of the drug substance originally contained in the multitude of the second strength is greater than the content of the drug substance originally contained in the first strength but lower than the supratherapeutic dose of the drug substance, and
c) a multitude of the second strength has released less than about 70% wt. of the overall content of the drug substance originally contained in the multitude of the second strength, wherein the overall content of the drug substance originally contained in the multitude of the second strength is greater than the supratherapeutic dose of the drug substance.

9. A pharmaceutical abuse deterrent composition containing particulates in which a release of a drug substance is controlled,
wherein the composition is constructed in more than one strength which comprises a first strength having a first acid neutralisation capacity and a second strength having a second acid neutralisation capacity, each of the first strength and the second strength individually contain an acid neutralising ingredient and the particulates of the drug substance, wherein
a. the second acid neutralisation capacity of the second strength is lower than the first acid neutralisation capacity of the first strength and
b. an amount of the particulates of the drug substance in the first strength is at least about 10% higher relative to the second strength,
wherein the first strength, the second strength and the multitude of the second strength when subjected to in-vitro dissolution testing for at least one corresponding time point:
a) difference between the drug substance release from the first strength and the drug substance release from the second strength is less than about 25,
b) difference between the drug substance release from the first strength and the drug substance release from a multitude of the second strength is less than about 25, wherein the overall content of the drug substance originally contained in the multitude of the second strength is greater than the content of the drug substance originally contained in the first strength but lower than the supratherapeutic dose of the drug substance, and
c) difference between the drug substance release from the first strength and the drug substance release from a multitude of the second strength is more than about 25, wherein the overall content of the drug substance originally contained in the multitude of the second strength is equal to or greater than the supratherapeutic dose of the drug substance,
wherein the drug substance released from the first strength, the second strength and the multitude of the second strength is measured in % wt. of the overall content of the drug substance originally contained in the first strength, the second strength and the multitude of the second strength, respectively.

10. A pharmaceutical abuse deterrent composition of claim 8 or claim 9, wherein the particulates of the drug substance in each of the first strength and the second strength comprising more than one matrix particulates, wherein the each matrix particulate comprising
a. a particulate comprising the drug substance and a pH dependent ingredient, wherein the drug substance is dispersed within matrix of the pH dependent ingredient, wherein the pH dependent ingredient is present in amount of not less than about 50% w/w of an amount of the drug substance and is at least one of
i. polyvinylacetal diethylamino acetate,
ii. copolymer of dimethylaminoethyl methacrylate, butyl methacrylate, methyl methacrylate, and
iii. copolymer of diethylaminoethyl methacrylate and methyl methacrylate, and
b. optionally an outer coating layer surrounding the particulate, wherein the outer coating layer comprising at least one of pH independent ingredient, water insoluble ingredient, surfactant, stabilizer, plasticizer and anti-tacking ingredient.

11. A pharmaceutical abuse deterrent composition of claim 8 or claim 9, wherein the particulates of the drug substance in each of the first strength and the second strength comprising a more than one coated particulates, wherein the each coated particulate comprising
a. a particulate comprising coat of the drug substance surrounding at least one of gelling ingredient, anti-crushing ingredient, pH independent ingredient, water insoluble ingredient and the particulate of aversion agent, and
b. an outer coating layer of the pH dependent ingredient surrounding the particulate, wherein the outer coating layer optionally further comprising at least one of pH independent ingredient, water insoluble ingredient, surfactant, stabilizer, plasticizer and anti-tacking ingredient, wherein the pH dependent ingredient is present in amount of not less than about 10% w/w of an amount of the drug substance and is at least one of
i. polyvinylacetal diethylamino acetate,
ii. copolymer of dimethylaminoethyl methacrylate, butyl methacrylate, methyl methacrylate, and
iii. copolymer of diethylaminoethyl methacrylate and methyl methacrylate.

12. A pharmaceutical abuse deterrent composition of claim 8 or claim 9, wherein the particulates of the drug substance in each of the first strength and the second strength comprising more than one coated particulates, wherein the each coated particulate comprising
a. a particulate comprising coat of the drug substance surrounding at least one of gelling ingredient, anti-crushing ingredient, pH independent ingredient, water insoluble ingredient and the particulate of aversion agent,
b. a functional coat surrounding the particulate and comprising at least one of pH independent ingredient, water insoluble ingredient, surfactant, stabilizer, plasticizer and anti-tacking ingredient, and
c. an outer coating layer of the pH dependent ingredient surrounding the functional coat, wherein the outer coating layer optionally further comprising at least one of pH independent ingredient, water insoluble ingredient, surfactant, stabilizer, plasticizer and anti-tacking ingredient, wherein the pH dependent ingredient is present in amount of not less than about 10% w/w of an amount of the drug substance and is at least one of
i. polyvinylacetal diethylamino acetate,
ii. copolymer of dimethylaminoethyl methacrylate, butyl methacrylate, methyl methacrylate and
iii. copolymer of diethylaminoethyl methacrylate and methyl methacrylate.

13. A pharmaceutical abuse deterrent composition of claim 8 or claim 9, wherein the particulates of the drug substance in each of the first strength and the second strength comprising more than one coated particulates, wherein the each coated particulate comprising
a. a particulate comprising the drug substance and at least one of pH dependent ingredient, pH independent ingredient, water insoluble ingredient, anti-crushing ingredient, and gelling ingredient, wherein the drug substance is dispersed within matrix of at least one of pH dependent ingredient, pH independent ingredient, water insoluble ingredient, anti-crushing ingredient, and gelling ingredient, and
b. the outer coating layer of the pH dependent ingredient surrounding the particulate, wherein the outer coating layer optionally further comprising at least one of pH independent ingredient, water insoluble ingredient, surfactant, stabilizer, plasticizer and anti-tacking ingredient, wherein the pH dependent ingredient is present in amount of not less than about 10% w/w of an amount of the drug substance and is at least one of
i. polyvinylacetal diethylamino acetate,
ii. copolymer of dimethylaminoethyl methacrylate, butyl methacrylate, methyl methacrylate and
iii. copolymer of diethylaminoethyl methacrylate and methyl methacrylate.

14. A pharmaceutical abuse deterrent composition of claim 8 or claim 9, wherein the particulates of the drug substance in each of the first strength and the second strength comprising more than one coated particulates, wherein the each coated particulate comprising
a. a particulate comprising the drug substance and at least one of pH dependent ingredient, pH independent ingredient, water insoluble ingredient, anti-crushing ingredient, and gelling ingredient, wherein the drug substance is dispersed within matrix of at least one of pH dependent ingredient, pH independent ingredient, water insoluble ingredient, anti-crushing ingredient, and gelling ingredient,
b. a functional coat surrounding the particulate and comprising at least one of pH independent ingredient, water insoluble ingredient, surfactant, stabilizer, plasticizer and anti-tacking ingredient, and
c. the outer coating layer of the pH dependent ingredient surrounding the functional coat, wherein the outer coating layer optionally further comprising at least one of pH independent ingredient, water insoluble ingredient, surfactant, stabilizer, plasticizer and anti-tacking ingredient, wherein the pH dependent ingredient is present in amount of not less than about 10% w/w of an amount of the drug substance and is at least one of
i. polyvinylacetal diethylamino acetate,
ii. copolymer of dimethylaminoethyl methacrylate, butyl methacrylate, methyl methacrylate and
iii. copolymer of diethylaminoethyl methacrylate and methyl methacrylate.

15. A pharmaceutical abuse deterrent composition of claim 8 or claim 9, wherein the particulates of the drug substance in each of the first strength and the second strength comprising more than one coated particulates, wherein the each coated particulate comprising
a. a particulate comprising a pharmaceutically acceptable derivative of the drug substance, and
a. the outer coating layer of the pH dependent ingredient surrounding the derivatives of drug substance, wherein the outer coating layer optionally further comprising at least one of pH independent ingredient, water insoluble ingredient, surfactant, stabilizer, plasticizer and anti-tacking ingredient, wherein the pH dependent ingredient is present in amount of not less than about 10% w/w of an amount of the drug substance and is at least one of
i. polyvinylacetal diethylamino acetate,
ii. copolymer of dimethylaminoethyl methacrylate, butyl methacrylate, methyl methacrylate and
iii. copolymer of diethylaminoethyl methacrylate and methyl methacrylate.

16. A pharmaceutical abuse deterrent composition of claim 8 or claim 9, wherein the particulates of the drug substance in each of the first strength and the second strength comprising more than one matrix particulates, wherein the each matrix particulate comprising
a. a particulate comprising a pharmaceutically acceptable derivative of the drug substance and a pH dependent ingredient, wherein the derivatives of drug substance is dispersed within matrix of the pH dependent ingredient, wherein the pH dependent ingredient is present in amount of not less than about 50% w/w of an amount of the drug substance and is at least one of
i. polyvinylacetal diethylamino acetate,
ii. copolymer of dimethylaminoethyl methacrylate, butyl methacrylate, methyl methacrylate, and
iii. copolymer of diethylaminoethyl methacrylate and methyl methacrylate.

17. A pharmaceutical abuse deterrent composition of claim 8 or claim 9, wherein the first strength and the second strength have resistance to alcohol dose dumping.

18. A pharmaceutical abuse deterrent composition of claim 8 or claim 9, wherein the acid neutralising ingredient in the first strength and the second strength is at least one of magnesium oxide, meglumine, sodium oxide, sodium hydroxide, sodium bicarbonate, sodium potassium tartrate, bismuth aluminate, bismuth carbonate, bismuth subcarbonate, bismuth subgallate, bismuth subnitrate, potassium citrate, sodium citrate, sodium carbonate, potassium bicarbonate, potassium carbonate, calcium carbonate, calcium phosphate, dibasic calcium phosphate, dihydroxyaluminumaminoacetate, dihydroxyaluminum sodium carbonate, glycine, magnesium glycinate, magnesium hydroxide, magnesium carbonate, sodium borate, aluminum oxide, aluminum hydroxide, ammonium carbonate, monoethanolamine, diethanolamine, triethanolamine, potassium hydroxide, calcium hydroxide, sodium phosphate dibasic, trolamine, sodium potassium tartrate, tribasic sodium phosphate and tricalcium phosphate.

19. A pharmaceutical abuse deterrent composition of claim 1, claim 8 or claim 9, wherein each of the first strength and the second strength individually further containing particulates of an aversion agent, wherein the aversion agent is not released in effective amount to produce its intended aversive effect unless the composition is administered after physical alteration but is released in effective amount to produce its intended aversive effect when the composition is administered after the physical alteration.

20. A pharmaceutical abuse deterrent composition of claim 19, wherein the aversion agent is present as coated particulates, wherein each coated particulate comprising
a. a particulate comprising the aversion agent, wherein the aversion agent is present
i. as a particle or
ii. within matrix of at least one ingredient of pH dependent ingredient, pH independent ingredient and water insoluble ingredient or
iii. as a coat surrounding at least one of pH dependent ingredient, pH independent ingredient and water insoluble ingredient, and
b. water insoluble coat surrounding the particulate.
